(19) United States
(12) Patent Application Publication
(10) Pub. No.: US 2006/0099645 A1
(43) Pub. Date: May 11, 2006

(54) Methods and reagents for assaying molecular interactions

(75) Inventors: Jeff Wrana, Toronto (CA); Miriam Barrios-Rodiles, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto (CA)

(21) Appl. No.: 10/526,733

(22) PCT Filed: Sep. 5, 2003
(86) PCT No.: PCT/CA03/01354

§ 371 Date: Aug. 15, 2005

(30) Foreign Application Priority Data
Sep. 6, 2002 (US) 60408922

(51) Int. Cl. G01N 33/53

(52) U.S. Cl. 435/7.1

(57) ABSTRACT

The invention relates to methods and reagents for screening, identifying, and/or quantifying molecular interactions. In particular, the invention provides a method for identifying protein-protein interactions comprising prey proteins interacting with bait proteins comprising: (a) introducing one or more prey protein in cells, wherein a prey protein is labelled with an epitope tag permitting separation of the prey protein from other proteins in the cells; (b) introducing one or more bait protein in the cells, wherein a bait protein is labelled with a detectable substance permitting detection of protein-protein interactions comprising a prey protein and the bait protein; and (c) assaying for protein-protein interactions comprising a prey protein and bait protein by detecting the detectable substance.

### FIELD OF THE INVENTION

The invention relates to methods and reagents for screening, identifying, and/or quantifying molecular interactions, in particular high throughput methods for assaying molecular interactions.

### BACKGROUND OF THE INVENTION

The activity of a cell and how it responds to its environment is governed by signal transduction pathways, which translate extracellular information into alterations in cell metabolism, trafficking, cell polarity, motility, shape, protein trafficking and gene expression. Moreover, pathological alterations in signal transduction pathways and cellular responsiveness to the extracellular environment underlie aspects of virtually every human disease. Much of the pioneering work in defining signal transduction pathways has focused on individual pathways and how specific components in these pathways function to elaborate intracellular signals. From these seminal studies has emerged the concept that formation of protein-protein complexes is a key molecular event that broadly underpins signal transduction pathways. Higher order protein complexes are formed by direct protein-protein interactions (PPIs) and during signal transduction their formation is positively and negatively regulated by post-translational modifications such as phosphorylation. PPIs can control activity by regulating enzymatic activity, substrate recognition, subcellular localization, inhibitor binding, membrane interactions and metabolite binding. There is considerable overlap in the use of specific components amongst different signaling cascades and recent work has revealed that cells likely respond to their environment not through linear signaling cascades but rather through complex networks of interacting proteins. However, current technology limits the scope of analysis to a relatively small number of components within these networks. Consequently it has not been possible to attain a genome-wide view of how the network of PPIs that are involved in signal transduction (the signal transduction interactome) functions to regulate cellular activity. The assembly of large arrays of sequenced, full length cDNA sets and the complete sequencing of the human genome, now provides key information and reagents with which to begin to define a signal transduction interactome.

In recent years, several techniques have been developed to facilitate large scale mapping of protein-protein interactions (PPIs). To date, the yeast two-hybrid system has been used almost exclusively for these projects and maps have been generated for the T7-bacteriophage, C. elegans S. cerevisiae and H. pylori genomes (Tucker, C. L., et al (2001). Trends Cell Biol. 11, 102-106). However, this approach has several limitations as it cannot be used for membrane-bound proteins or transcriptional co-regulators. At present, a systematic large-scale analysis of PPIs within mammalian cells has not yet been published, however, several methods are being developed. Fluorescent imaging approaches in which protein interactions are detected by the fluorescence resonance energy transfer (FRET) that occurs when two proteins come in close proximity to one another are being developed (Pollok, B. A., and Heim, R. (1999). Trends Cell Biol. 9, 57-60. The primary limitation of this approach is that the fluorescent tags must be sufficiently close to permit energy transfer. Other approaches have utilized protein-fragment complementation assays (PCA; reviewed in Remy, I., and Michnick, S. W. (2001). Proc. Natl. Acad. Sci. (USA) 98, 7678-7683 and Michnick, S. W. (2001). Curr. Op. Struct. Biol. 11, 472-477). For this, a reporter enzyme is ‘split’ into two complementary fragments each of which is fused to proteins of interest. When two proteins associate they bring together the complementing proteins and restore enzymatic activity. Both β-galactosidase and dihydrofolate reductase (DHFR) have been used in PCAs using model protein interaction networks (Remy, I., and Michnick, S. W. (2001). Proc. Natl. Acad. Sci. (USA) 98, 7678-7683 16; Michnick, S. W. (2001). Curr. Op. Struct. Biol. 11, 472-477; and Rossi, F., Chariton, C. A., and Blau, H. M (1997). Proc. Natl. Acad. Sci. USA 94, 8405-8410). PCA provides a powerful tool to visualize PPIs in mammalian cells, however it is unclear whether the current approaches and sensitivity are amenable to high throughput (HTP) screening. Considerable effort has also been directed towards mass spectrometric approaches for large-scale analysis of protein samples (Figeys, D., McBroom, L. D., and Moran, M. F. (2001). Methods 24, 230-239). For this, proteins of interest and their interacting partners are isolated using high-affinity antibodies, separated by gel electrophoresis, subjected to trypsin digestion and then identified by mass spectrometry. This technique is highly dependent on obtaining sufficient quantities of high quality tryptic peptides and on appropriate proteomic databases to provide unambiguous protein identification.

The citation of any reference herein is not an admission that such reference is available as prior art to the instant invention.

### SUMMARY OF THE INVENTION

The present invention makes available a rapid, effective assay and reagents for screening, identifying, and/or quantitating molecular interactions, or components thereof, and the fluctuation of such interactions in response to stimulation and the environment. The subject assay enables rapid screening of large numbers of molecules to identify interactions, and agents that affect such interactions. The invention contemplates reagents and methods to screen, identify, and/or quantitate molecular interactions.

Molecular interactions include but are not limited to interactions involving proteins, nucleic acids, and ligands. In particular, molecular interactions may involve protein-protein interactions associated with signal transduction pathways.

In an aspect an assay and reagents of the invention are used to screen, identify, and/or quantitate protein-protein interactions. In particular, an assay of the invention may be characterized by the use of reagent or recombinant cells to identify polypeptides that interact with one or more bait protein. In an aspect, reagent or recombinant cells are used to sample a polypeptide library for polypeptides that interact with one or more bait protein. As described with greater detail below, the recombinant or reagent cells express one or more bait protein capable of transducing a detectable signal in the reagent cell, and prey proteins for which interaction with a bait protein is to be ascertained. Collectively, a mixture of such reagent or recombinant cells provides a variegated library of potential proteins that interact with one or more bait protein. Members of the library which interact with a bait protein can be selected and identified.

Therefore, the invention contemplates a recombinant cell, in particular a mammalian cell, comprising:
(a) an expressable recombinant vector encoding a prey protein and an epitope tag permitting separation of the prey protein; and
(b) an expressible recombinant vector encoding a bait protein and a detectable substance that permits detection of protein-protein interactions comprising the prey protein and bait protein.

In an aspect the invention provides a mixture of recombinant cells of the invention.

In another aspect, recombinant cells of the invention comprise recombinant vectors encoding two or more bait proteins. In an embodiment, each bait protein is labeled with a different detectable substance to facilitate detection of protein-protein interactions comprising a bait protein and prey protein.

In a further aspect of the invention, the recombinant cells comprise recombinant vectors encoding two or more prey proteins.

The signal transduction activity of a prey and/or bait protein in recombinant cells or in cells in a mixture of recombinant cells may be modulated by an intracellular or extracellular signal.

The invention also provides a gene library comprising a mixture of nucleic acid molecules comprising sequences encoding a variegated population of prey proteins involved in signal transduction pathways or cell cycle pathways. The invention also contemplates a polypeptide library encoded by a gene library of the invention. A polypeptide library of the invention generally comprises a variegated population of prey proteins involved in signal transduction pathways or cell cycle pathways.

A recombinant or reagent cell or mixture thereof, or gene or protein library of the invention, may be used to identify protein-protein interactions, and agents that affect such interactions. Protein-protein interactions that lead to cell behaviour or gene responses may be identified by the methods of the invention.

Therefore, the invention provides a system for assaying for protein-protein interactions, and agents that affect such interactions comprising reagent or recombinant cells or a mixture of reagent or recombinant cells, or a gene or protein library of the invention.

In an aspect, the invention provides a method for identifying prey proteins that interact with one or more bait protein comprising:
(a) introducing, one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting detection of protein-protein interactions comprising a prey protein and the bait protein; and
(c) assaying for protein-protein interactions comprising a prey protein and a bait protein by detecting the detectable substance.

The invention also relates to methods for quantitating protein-protein interactions.

The invention further relates to a method for determining an interactome for a proteome comprising identifying protein-protein interactions using a method of the invention and determining the interactome based on the protein-protein interactions.

In an embodiment, a method is provided for determining an interactome for a proteome comprising:
(a) preparing a mixture of recombinant cells expressing one or more bait protein from the proteome, and one or more prey protein selected from a variegated population of prey proteins;
(b) inducing formation of protein-protein interactions between a prey protein and bait protein in the cells;
(c) identifying protein-protein interactions comprising a prey protein and bait protein; and
(d) determining the interactome based on the identified protein-protein interactions.

The invention also relates to a method for determining the function of a gene product comprising:
(a) defining an interactome of the gene product comprising:
   (i) preparing a mixture of recombinant cells expressing the gene product and one or more prey protein selected from a variegated population of prey proteins, and
   (ii) identifying protein-protein interactions comprising the gene product and a prey protein to define an interactome;

and
(b) determining the function of the gene product based on the structure and/or function of prey proteins that interact with the gene product in the interactome.

The invention also relates to a method for determining a disease or condition associated with a test protein comprising:
(a) defining an interactome for the test protein comprising:
   (i) preparing recombinant cells expressing the test protein and one or more prey proteins selected from a variegated population of prey proteins, and
   (ii) identifying protein-protein interactions comprising the test protein and a prey protein to define an interactome for the test protein;

and
(b) determining a disease or condition associated with the test protein based on the identity of the proteins that interact with the test protein in the interactome.

The methods of the invention may further comprise a clustering step to identify protein-protein interactions that have similar dynamics and/or behaviour and thus may function as a coordinated response.

The invention also relates to methods for systematically analyzing protein-protein interactions in cell signaling, and methods for analyzing protein-protein interactions in different cell types. The invention also provides methods for assaying for changes in protein-protein interactions in response to intracellular or extracellular factors.

The invention permits the identification of agents or compounds that interact with and modulate the activity of a protein-protein interaction or component thereof and are potentially useful as therapeutics. Thus, the present invention provides a convenient format for discovering drugs that can be useful to modulate cellular function, as well as to understand the pharmacology of agents or compounds that specifically modulate protein-protein interactions.

In an aspect the invention provides a method for evaluating a compound for its ability to modulate a signal transduction pathway through a prey protein, bait protein, or protein-protein interaction of the invention. For example, the compound may be a substance which binds to a prey protein, bait protein, or protein-protein interaction, or which disrupts or promotes the interaction of proteins in a protein-protein interaction.

The invention also provides a method for identifying an agent to be tested for the ability to modulate a signal transduction pathway by testing for the ability of the agent to affect the interaction between the molecules in a protein-protein interaction, wherein the protein-protein interaction is part of the signal transduction pathway.

In an embodiment the invention provides a method for identifying a potential modulator of signal transduction activity.

Another aspect of the present invention provides a method of conducting a drug discovery business comprising:
(a) conducting therapeutic profiling of agents identified in accordance with a method of the invention, or further analogs thereof, for efficacy and toxicity in animals; and
(b) formulating a pharmaceutical preparation including one or more agents identified in step
   (a) as having an acceptable therapeutic profile.

Yet another aspect of the invention provides a method of conducting a target discovery business comprising licensing, to a third party, the rights for further drug development and/or sales for agents identified in accordance with a method of the invention, or analogs thereof.

The methods of the invention may be used generally to detect mutations in cellular proteins that disrupt protein-protein interactions.

The methods of the invention can also be used in the form of a diagnostic assay to detect the interaction of two proteins, for example, where the protein or gene encoding same is isolated from biopsied cells.

The invention also provides methods for constructing a protein linkage map for a proteome or interactome.

The invention also contemplates a matrix comprising a color gradient displaying the magnitude of one or more protein-protein interactions identified using a method of the invention.

The invention provides libraries of information on protein-protein interactions, methods to construct such libraries, and data sharing systems which enable efficient utilization of such libraries. Furthermore, the invention provides databases which accommodate and maintain libraries of information relative to such protein-protein interactions, methods and systems to construct such databases, methods and systems to enable a client to search through such databases for desired information, methods and systems to transmit to a client desired pieces of information concerning protein-protein interactions that are housed in databases, tangible electronic means to record and make use of such systems and databases, and apparatus to enable construction and search of databases and/or transmission of desired information to a client.

The methods of the invention can be carried out in a high throughput format. In drug screening programs which test libraries of compounds and natural extracts, high throughput assays are desirable in order to maximize the number of compounds screened in a given period of time.

The identification of protein-protein interactions and active compounds within libraries using the methods described herein can be followed by other identification procedures, for example, mass spectroscopy.

The invention also provides an integrated modular system for performing the methods of the invention.

The methods of the present invention, as described above, may be practiced using kits for detecting and characterizing interactions between a bait protein and one or more prey proteins.

The invention also encompasses the agents/compounds identified using a method of the invention.

The agents/compounds identified using the methods of the invention may be formulated into compositions for administration to individuals suffering from a disease or condition. Therefore, the present invention also relates to a composition comprising one or more of an agent/compound identified using a method of the invention, and a pharmaceutically acceptable carrier, excipient or diluent. A method for modulating a signal transduction activity associated with a disease or condition is also provided comprising introducing into the cells an agent/compound identified using a method of the invention or a composition containing same.

Still further the invention provides the use of an agent/compound identified using a method of the invention in the preparation of a medicament to treat individuals suffering from a disease or condition.

The disruption or promotion of the interaction between the molecules in protein-protein interactions identified using a method of the invention is useful in therapeutic procedures. Therefore, the invention features a method for treating a subject or individual having a disease or condition characterized by an abnormality in a signal transduction pathway wherein the signal transduction pathway involves an interaction between a prey protein and a bait protein.

In yet another aspect the invention provides a method of treating diseases or conditions where the affected cells have a defective prey or bait protein (e.g. mutated target protein or over expressed target protein) comprising administering an effective amount of an agent or compound identified using a method of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

### DESCRIPTION OF THE DRAWINGS

The invention will be better understood with reference to the drawings in which:

FIG. 1 is a schematic of a screen to assay protein-protein interactions in mammalian cells. Renilla luciferase (Rluc) fused to the bait protein is coexpressed with epitope (flag)-tagged proteins and the cells stimulated to induce formation of protein complexes. Flag-tagged protein is purified on magnetic affinity resins and co-purified Rluc-tagged bait protein is detected enzymatically.

FIG. 2 shows the application of luciferase fusions to analysis of protein-protein interactions. Smad4-Rluc was transiently expressed in 293T cells either alone or together with flag-tagged wild type Smad2 (F-S2), a phosphorylation site mutant of Smad2 (F-S2(2SA)) or Smad1, as indicated. Formation of R-Smad-Smad4-Rluc complexes in the absence and presence of TGFβ (left panel) or BMP signalling (right panel) was assayed as diagrammed in the schematics.

FIG. 3 shows an analysis of the TGFβ signal transduction interactome. The interactome of Smad4 and TβRI assayed against 40 cDNAs is shown. Each square is the mean of three assays. Smad4 was assayed in the presence and absence of TGFβ signalling. In addition, kinase-deficient (KR) and constitutively active activated TGFβ type I receptors were screened against the set. Quantitation of the interactions is visualized colorimetrically using the scale shown below. Note the strong TGFβ-dependent interaction of Smad4 with R-Smad2 and 3 and the signalling-independent interaction with Ski. In the case of the type I receptor, known interactors are labelled as well as novel interactions detected in this screen (asterisks).

### DETAILED DESCRIPTION OF THE INVENTION

Glossary

Certain terms employed in the specification, examples, and appended claims are, for convenience, collected here.

As used herein, “recombinant cells” include any cells that have been modified by the introduction of heterologous nucleic acids (e.g. DNA). Suitable cells can be found in Goeddel, Gene Expression Technology: Methods in Enzymology 185, Academic Press, San Diego, Calif. (1991), and include a wide variety of eukaryotic host cells, preferably mammalian cells.

“Heterologous nucleic acid” or “heterologous DNA” includes nucleic acids (in particular DNA) that does not occur naturally as part of the genome in which it is present, or which is found in a location(s) in the genome that differs from that in which it occurs in nature. A heterologous nucleic acid is not endogenous to the cell into which it is introduced, but has been obtained from another cell. Generally, although not necessarily, such nucleic acid encodes RNA and proteins that are not normally produced by the cell in which it is expressed. A heterologous nucleic acid may also be referred to as foreign nucleic acid. The term encompasses any nucleic acid that one of skill in the art would recognize or consider as heterologous or foreign to the cell in which it is expressed. Examples of heterologous DNA include, but are not limited to, DNA that encodes a prey protein, bait protein, or test polypeptide.

“Bait protein” refers to a protein which is to be tested for interaction with a prey protein. Generally, the bait protein comprises all or part of a target molecule which has either been implicated in a biological process of interest or for which the function is sought. Suitable bait proteins include functional domains of a wide variety of proteins involved in signal transduction, including, but not limited to, receptors, ligands, hormones, enzymes, transcription proteins, cell cycle proteins, etc. A bait protein may also be a random protein. For example, the protein may be from about 2 amino acids to about 100 amino acids. In an embodiment, a bait protein is fully randomized, with no sequence preferences or constants at any position. In another embodiment, the protein is biased and some positions within the sequence are held constant, or are selected from a limited number of possibilities. By way of example, nucleotides or amino acid residues may be randomized within a defined class including hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, or to reduce the chance of creation of a stop codon, etc.

In a preferred embodiment, the bias is towards proteins or nucleic acids that interact with known classes of molecules. For example, intracellular signaling is carried out via short regions of polypeptides interacting with other polypeptides through small peptide domains. For instance, short SH2 and SH3 target peptides have been used as pseudosubstrates for specific binding to SH2 proteins and SH3 proteins respectively. This is just an example of available peptides with biological activity, as there is an abundance of literature in this area. In addition, agonists and antagonists of signaling molecules may be used as the basis of biased randomization of bait proteins.

In an embodiment, the bait protein is a protein associated with signal transduction pathways or cell cycle pathways. In a particular embodiment, the bait proteins possess domains known to be involved in signal transduction pathways. The bait proteins may have known or unknown function. In particular embodiments, the bait proteins are proteins of the TGFβ proteome (e.g. Smad proteins, SARA family proteins, Smad-interacting proteins, TGFβ receptors, and receptor interacting proteins, SMURFs, BMP receptors), the WNT pathway (e.g. APC, β-catenin, axin, dishevelled, GSK-3β, and TCFs1-4), Sak/Polo pathway (e.g. Sak, Plks) or receptor tyrosine kinase pathways (e.g. EGF, FGF, PDGF, NGF).

In another embodiment, the bait protein is a protein with unknown function that has been associated with disease. Examples of these proteins include LKB1, TUBEROUS SCLEROSIS 1 and 2 (TSC1 and TSC2), and POLYCYSTIC KIDNEY DISEASE 1 and 2 (PKD1 and PKD2).

“Prey protein” refers to a candidate protein that is to be tested for interaction with a bait protein. In an embodiment, the prey protein is one of a library of protein sequences or polypeptide library (i.e. a library of prey proteins is tested for binding to one or more bait proteins). The prey protein sequences can be obtained from genomic DNA, cDNA or can be random sequences. Specific classes of prey proteins may also be tested. A library of prey proteins or sequences encoding prey proteins may be incorporated into a library of vectors, each or most containing one or more different prey protein sequence.

In an embodiment, the prey protein sequences are obtained from genomic DNA sequences. Genomic digests may be cloned into recombinant vectors. A genomic library may be a complete library, or it may be fractionated or enriched.

In another embodiment, the prey protein sequences are obtained from cDNA libraries. A cDNA library from any number of different cells or organisms may be used, and cloned into test vectors. A cDNA library may be a complete library, or it may be fractionated or enriched.

Prey protein sequences may be random sequences. These are generally generated from chemically synthesized oligonucleotides. Generally, random prey proteins range in size from about 2 amino acids to about 100 amino acids. Fully random or “biased” random proteins may be used as described herein.

Bait proteins are preferably fused to a detectable substance and prey protein(s) are preferably fused to an epitope tag, as described further below. However, as will be appreciated by those in the art, the bait proteins may be fused to the epitope tag, and the prey proteins may be fused to the detectable substance.

“Detectable substance” refers to a substance for labeling a bait protein that permits detection of the bait protein. Suitable detectable substances include, but are not limited to, radioisotopes (e.g. ¹H, ⁴C, ³S, ¹²⁵I, ³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), and biotinyl groups (which can be detected by marked avidin e.g., streptavidin containing a fluorescent marker or enzymatic activity that can be detected by optical or colorimetric methods). Optimal results are obtained if an enzymatic detectable substance is employed in the present invention. In a preferred aspect of the invention, the detectable substance is a luciferase, more preferably Renilla luciferase.

“Epitope tag” refers to a marker that allows for efficient recovery of a tagged protein (e.g. prey protein) from cell lysates, preferably mammalian cell lysates. A suitable epitope tag is a FLAG peptide that can be used as an epitope tag in many cell types. The sequence, use and detection of the FLAG tag is described in Chubet, RG, et al. (Biotechniques January 1996; 20(1):136-41). Vectors for expression and secretion of FLAG epitope-tagged proteins in mammalian cells is described in Biotechniques January 1996 20(1):136-41. Other epitope tags include the hemagglutinin (“HA”) tag, His6, or Ig sequence.

The terms “protein”, “polypeptide” and “peptide” are used interchangeably and refer to a sequence of amino acids of any length, constituting all or a part of a native-sequence or naturally-occurring polypeptide or peptide, or constituting a non-naturally-occurring polypeptide or peptide (e.g., a randomly generated peptide sequence or one of an intentionally designed collection of peptide sequences).

A bait protein or prey protein includes but is not limited to native-sequence polypeptides, and isoforms, chimeric polypeptides, homologs, or fragments of a native-sequence polypeptide. A “native-sequence polypeptide” comprises a polypeptide having the same amino acid sequence of a polypeptide derived from nature. The term also encompasses truncated or secreted forms of a polypeptide, polypeptide variants including naturally occurring variant forms (e.g. alternatively spliced forms or splice variants), naturally occurring allelic and species variants, and analogs.

The term “polypeptide variant” means a polypeptide having at least about 70-80%, preferably at least about 85%, more preferably at least about 90%, most preferably at least about 95% amino acid sequence identity with a native-sequence polypeptide Such variants include, for instance, polypeptides wherein one or more amino acid residues are added to, or deleted from, the N— or C-terminus of a full-length or mature sequence including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. A naturally occurring allelic variant may contain conservative amino acid substitutions from the native polypeptide sequence.

The term “polypeptide library” or “library of protein sequences” is used herein to indicate a variegated ensemble of polypeptide sequences, where the diversity of the library may result from cloning, mutagenesis, or random or semi-random synthesis of nucleic acid sequences. In an embodiment, the polypeptide library is a variegated ensemble of prey proteins. The term “gene library” has a similar meaning, indicating a variegated ensemble of nucleic acid molecules.

The term “nucleic acid” is intended to include two or more nucleotides covalently bonded together such as deoxyribonucleic acid (DNA) or ribonucleic acids (RNA) and including, for example, single-stranded and a double-stranded nucleic acid. It is intended to include, for example, genomic DNA, cDNA, mRNA and synthetic oligonucleotides corresponding thereto which can represent the sense strand, the anti-sense strand or both. A nucleic acid can include natural and non-naturally occurring modifications such as post-transcriptional modifications, minor substitutions and incorporation of functionally equivalent nucleotide analogs and mimetics. Such changes and methods of incorporation are well known to those skilled in the art.

The terms “interact”, “interaction”, or “interacting” refer to any physical association between proteins, other molecules such as lipids, carbohydrates, nucleotides, and other cell metabolites. Examples of interactions include protein-protein interactions. The term preferably refers to a stable association between two molecules due to, for example, electrostatic, hydrophobic, ionic and/or hydrogen-bond interactions under physiological conditions. Certain interacting or associated molecules interact only after one or more of them has been stimulated (e.g. phosphorylated). An interaction between proteins and other cellular molecules may be either direct or indirect.

“Extracellular signal” or “extracellular factor” includes a molecule or a change in the environment that is transduced intracellularly via cell surface proteins (e.g. cell surface receptors) that interact, directly or indirectly, with the signal. An extracellular signal includes any compound or substance that in some manner specifically alters the activity of a cell surface protein. Examples of such signals or factors include, but are not limited to growth factors and hormones, that bind to cell surfaces and/or intracellular receptors and ion channels and modulate the activity of such receptors and channels. The signals and factors include analogs, derivatives, mutants, and modulators of such growth factors and hormones.

“Intracellular signal” or “intracellular factor” includes a molecule or a change in the cell environment that is transduced in the cell via cytoplasmic proteins that interact, directly or indirectly with the signal. An intracellular signal includes any compound or substance that in some manner specifically alters the activity of a cytoplasmic protein involved in a signal transduction pathway.

“Signal transduction” refers to the process of signaling from the cellular environment through the cell membrane, and may occur through one or more of several mechanisms, such as phosphorylation, activation of ion channels, effector enzyme activation via guanine nucleotide binding protein intermediates, formation of inositol phosphate, activation of adenyl cyclase, and/or direct activation (or inhibition) of a transcriptional factor.

“Signal transduction pathway” refers to the sequence of events that involves the transmission of a message from an extracellular protein to the cytoplasm through the cell membrane. Signal transduction pathways contemplated herein include pathways involving a regulatory protein or motif, or protein-protein interactions or an interacting molecule thereof. The methods of the invention may be used to assay the amount and intensity of a given signal in a signal transduction pathway.

The present invention can be applied to signal transduction pathways that regulate important aspects of cellular activity and have well-described core components that provide an important framework from which dynamic signal transduction interactome maps can be built. Examples of particular signaling transduction pathways include the TGFβ signalling pathway, the Wingless pathway, receptor tyrosine kinase (RTK) pathways, and pathways associated with polo kinases.

The TGFβ signalling pathway plays critical roles in a wide range of developmental processes and human diseases. The transforming growth factor-β family represents a large group of secreted polypeptide growth and differentiation factors (Attisano, L., and Wrana, J. L. (2000). Curr. Op. Cell Biol. 12, 235-243; Wrana, J. L., and Attisano, L. (2000). Cyto. Growth Factor Rev. 11, 5-13; and Massagué, J., Blain, S. W., and Lo, R. S. (2000). Cell 103, 295-309). These proteins regulate aspects of virtually all developmental and homeostatic processes and aberrant activity of this pathway is associated with numerous human diseases. TGFβ family members signal through heteromeric ser/thr kinase receptor complexes in which the type II receptor phosphorylates the type I receptor and activates it to transmit signals to the downstream Smad signal transduction pathway. Receptors activate Smad signalling by directly phosphorylating receptor-regulated Smads (R-Smads) that are recruited to membranes through the anchoring protein, SARA. Kinase cascades such as p38 and JNK can also be activated. Phosphorylation of R-Smad induces it to form a heteromeric complex with the common Smad, Smad4 and drives translocation of the R-Smad-Smad4 complex into the nucleus, where it regulates transcription through R-Smad-mediated interaction with DNA binding partners. Activated R-Smads can also regulate protein stability by mediating interaction of Smurf ubiquitin ligases with protein targets. Thus, Smads translate TGFβ signals into alterations in gene expression and protein stability through protein-protein interactions that are regulated by phosphorylation and subcellular localization. Therefore, defining an interactome for this pathway will lead to an understanding of how TGFβ regulates biological responses.

The Wingless pathway, which crosstalks with Smads, is regulated by ubiquitin-dependent proteolysis and is mutated in colorectal carcinoma in humans. Wnt/wingless signalling pathway plays a pivotal role in many developmental processes including cell differentiation, migration, proliferation and cell polarity (Cadigan, K. M., and Nusse, R (1997). Genes Dev. 11, 3286-3305; and Kuhl, M., Sheldahl, L. C., Park, M., Miller, J. R., and Moon, R. T. (2000). Trends Genet. 16, 279-283) and activation of this pathway has been linked to tumorigenesis. Wnt signalling through the ‘canonical’ pathway regulates the intracellular effector, β-catenin (Cadigan, K. M., and Nusse, R. (1997). Genes Dev. 11, 3286-3305). A multiprotein complex including adenomatous polyposis coli (APC) and axin family proteins facilitates GSK3-dependent phosphorylation of β-catenin which induces ubiquitin-dependent degradation of β-catenin. Binding of Wnt to the Frizzled family of transmembrane receptors leads to inhibition of GSK activity-through a mechanism involving the Dishevelled protein. This blocks β-catenin degradation allowing it to accumulate and enter the nucleus where it binds LEF/TCF transcription factors and activates specific target genes. Several Wnt ligands (Wnt-1, 3A, 8 and 8B) signal through this pathway. However, other Wnts (such as Wnt-4, 5A and 11) regulate distinct cellular and embryonic responses through the non-canonical pathway that involves intracellular calcium release and activation of PKC and Ca2+-Calmodulin kinase II (Kuhl, M., Sheldahl, L. C., Park, M., Miller, I. R, and Moon, R. T. (2000). Trends Genet. 16, 279-283). The molecular components of this response may involve G proteins. Selection between these two pathways appears to be determined at the receptor level as distinct frizzled receptors preferentially activate either the β-catenin or Ca²⁺ pathway.

Signal transduction pathways mediated by receptor tyrosine kinases (RTK) and protein tyrosine kinase (PTKs) involve integration and amplification of multiple extracellular and intracellular signals by second messengers, and the activation of cellular processes including cell proliferation, cell division, cell growth, the cell cycle, cell differentiation, cell migration, axonogenesis, nerve cell interactions, and regeneration. Signaling pathways mediated by receptor tyrosine kinases may be initiated by growth factors binding to specific receptors on cell surfaces. One such growth factor is epidermal growth factor (EGF) which induces proliferation of a variety of cells in vivo. The binding of EGF to its receptor (epidermal growth factor receptor—EGFR) activates a RTK/PTK signaling pathway. The EGF receptor has an extracellular N-terminal domain that binds EGF and a cytoplasmic C-terminal domain containing an EGF-dependent protein tyrosine kinase that is capable of autophosphorylation and the phosphorylation of other protein substrates. The binding of EGF to its receptor activates the tyrosine kinase which phosphorylates a variety of signaling molecules thereby initiating a RTK/PTK signaling pathway that leads to DNA replication, RNA and protein synthesis, and cell division. Other RTK/PTK signaling pathways can be activated through the following receptor tyrosine kinases: PDGFR, insulin receptor tyrosine kinase, Met receptor tyrosine kinase, fibroblast growth factor (FGF) receptor, insulin receptor, insulin growth factor (IGF-1) receptor, TrkA receptor, IL-3 receptor, B cell receptor, TIE-1, Tek/Tie2, Flt-1, Flk, VEGFR3, EFGR/Erbb, Erb2/neu, Erb3, Ret, Kit, Alk, Axl, FGFR1, FGFR2, FGFR3, keratinocyte growth factor (KGF) receptor, EphA receptors including but not limited to EphA1 (also known as Eph and Esk), EphA2 (also known as Eck, Myk2, Sek2), EphA3 (also known as Cek4, Mek4, Hek, Tyro4, Hek4), EphA4 (also known as Sek, Sek1, Cek8, Hek8, Tyro1), EphA5 (also known as Ehk1, Bsk, Cek7, Hek7, and Rek7), EphA6 (Ehk2, and Hek12) EphA7 (also known as Mdk1, Hek11, Ehk3, Ebk, Cek11), and EphA8 (also known as Eek, Hek3); and the Eph B receptors including but not limited to EphB1 (also known as Elk, Cek6, Net, Hek6), EphB2 (also known as Cek5, Nuk, Erk, Qek5, Tyro5, Sek3, hek5, Drt), EphB3 (also known as Cek10, Hek2, Mdk5, Tyro6, and Sek4), EphB4 (also known as Htk, Myk1, Tyro11, Mdk2), EphB5 (also known as Cek9, Hek9), and EphB6 (also known as Mep).

Protein tyrosine kinases (i.e. intracellular tyrosine kinases) include members of the Src family including Src, Fyn, Yes, Lyn, Lck, Yrk, Hrk, and Blk; members of the BTK family including BTK, Tec, and Itk; members of the Jak family including Jak1, Jak2, and Jak3; and Abl, Fak, Zap70, Syk, Tyk, Fer, Fes, Csk, Ntk, Pyk.

The term “modulation of a signal transduction activity” in its various grammatical forms, as used herein, includes induction and/or potentiation, as well as inhibition of one or more signal transduction pathways.

“Modulators” refers to substances that modulate a protein-protein interaction (e.g. an interaction between a bait and a prey protein), to thereby modulate a signal transduction activity or pathway and influence cellular functions. Such substances are potential pharmacological agents that may be used to treat diseases by modulating the activity of specific protein-protein interactions or signal transduction pathways. A modulator may inhibit or potentiate a protein-protein interaction and it may be an agonist or antagonist. A modulator may modulate signal transduction via a receptor by binding to the receptor, though not necessarily at the binding site of the natural ligand. A modulator may modulate signal transduction when used alone, or can alter signal transduction in the presence of the natural ligand, either to enhance or inhibit signaling by the natural ligand.

“Antagonists” are molecules that block or decrease the signal transduction activity, e.g., they can competitively, noncompetitively, or allosterically inhibit signal transduction. “Agonists” potentiate, induce or otherwise enhance the signal transduction activity.

“Disease” or “condition” refers to a state that is recognized as abnormal by the medical community. The disease or condition may be characterized by an abnormality in a signal transduction pathway in a cell wherein one of the components of the signal transduction pathway is a regulatory protein or sequence motif thereof.

“Abnormality” or “abnormal” refers to a level which is statistically different from the level observed in organisms not suffering from a disease or condition. It may be characterized by an increased amount, intensity or duration of signal, or a deficient amount, intensity or duration of signal. An abnormality may be realized in a cell as an abnormality in cell function, viability, or differentiation state. An abnormal protein-protein interaction level may be greater or less than a normal level and may impair the performance or function of an organism.

“Interactome” refers to a network or set of protein-protein interactions particularly protein-protein interactions that are involved in signal transduction that function to regulate cellular activity. An “interactome” may be defined as the entire interaction map of a proteome—analogous to a wiring diagram or schematic, specifying the entire signal transduction and metabolic networks of the cell.

“Proteome” refers to the entire complement of proteins specified by a genome, or expressed by a given tissue or cell type. A proteome may refer to a complement of proteins expressed by a given tissue or cell type of a subject, in particular a diseased tissue or cell of a subject.

Reagents

Cells and Vectors

The invention contemplates a reagent or recombinant cell, in particular a mammalian cell, comprising:
(a) an expressable recombinant vector encoding one or more prey protein and an epitope tag permitting separation of the prey protein; and
(b) an expressible recombinant vector encoding one or more bait protein and detectable substance that permits detection of protein-protein interactions comprising a prey protein and bait protein.

The invention also contemplates a recombinant cell, in particular a mammalian cell comprising:
(a) an expressable recombinant vector encoding one or more prey protein and an epitope tag permitting separation of the prey protein; and
(b) an expressible recombinant vector encoding one or more bait protein and detectable substance that permits detection of protein-protein interactions comprising a prey protein and bait protein;
   and wherein the signal transduction activity of one or both of a prey protein and bait protein is modulated by an intracellular or extracellular signal.

In an aspect the invention provides a mixture of recombinant cells, in particular mammalian cells, each of which comprises:
(a) an expressable recombinant vector encoding one or more prey protein and an epitope tag permitting separation of the prey protein; and
(b) an expressible recombinant vector encoding one or more bait protein and detectable substance that permits detection of protein-protein interactions comprising a prey protein and bait protein;
   wherein collectively the mixture of cells expresses a variegated population of prey proteins.

The signal transduction activity of a prey and/or bait protein in recombinant cells of the invention or cells in a mixture of recombinant cells may be modulated by an intracellular or extracellular signal. The intracellular or extracellular signal may be a protein that is introduced into the cell by an expressable recombinant vector encoding the protein, or one of the expressable recombinant vectors encoding the prey protein or bait protein may encode the protein.

A recombinant cell of the invention may also comprise an expressable recombinant vector encoding a protein required for inducing signal transduction in the cells involving the interaction of the bait and prey proteins. For example, a recombinant cell may also include an expressable recombinant vector encoding a receptor (e.g. TGFβ receptor, receptor tyrosine kinase, etc.), or adaptor protein (e.g. Grb2, Shc, etc.). Alternatively, one of the expressable recombinant vectors encoding the prey protein or bait protein may additionally encode the protein.

A recombinant vector encoding one or more prey protein or bait protein may be prepared using conventional methods. Nucleic acids which encode prey or bait proteins may be incorporated in a known manner into an appropriate expression vector which ensures good expression of the proteins. Possible expression vectors include but are not limited to cosmids, plasmids, or modified viruses so long as the vector is compatible with the host cell used. The expression vectors contain a nucleic acid encoding the protein and the necessary regulatory sequences for the transcription and translation of the inserted protein sequence. Suitable regulatory sequences may be obtained from a variety of sources, including bacterial, fungal, viral, mammalian, or insect genes. [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen, and may be readily accomplished by one of ordinary skill in the art. Other sequences, such as an origin of replication, additional DNA restriction sites, enhancers, and sequences conferring inducibility of transcription may also be incorporated into the expression vector.

Appropriate vectors for use with mammalian cellular hosts are known in the art, and are described in, for example, Powels et al. (Cloning Vectors: A Laboratory Manual, Elsevier, New York, 1985). Mammalian expression vectors may contain both prokaryotic sequences, to facilitate the propagation of the vector in bacteria, and one or more eukaryotic transcription units that are expressed in eukaryotic cells. Examples of mammalian vectors suitable for transfection of eukaryotic cells include the pcDNAI/amp, pcDNAI/neo, pRc/CMV, pSV2gpt, pSV2neo, pSV2-dhfr, pTk2, pRSVneo, pMSG, pSVT7, pCMV5C, pko-neo and pHyg derived vectors. Some of these vectors may be modified with sequences from bacterial plasmids (e.g. pBR322), to facilitate replication and drug resistance selection in both prokaryotic and eukaryotic cells.

Transcriptional and translational regulatory sequences in vectors to be used in transforming mammalian cells may be provided by viral sources. Promoters and enhancers may be derived from viruses such as Polyoma, Adenovirus 2, Simian Virus 40 (SV40), and human cytomegalovirus. DNA sequences derived from the SV40 viral genome, (e.g. SV40 origin, early and late promoter, enhancer, splice, and polyadenylation sites) may be used to provide the control elements required for expression of a heterologous DNA sequence. Derivatives of viruses such as the bovine papillomavirus (BPV-1), or Epstein-Barr virus (pHEBo, pREP-derived and p205) may be used for transient expression of proteins in eukaryotic cells.

The recombinant vectors may also contain nucleic acids which encode a portion which provides increased expression of the recombinant protein; increased solubility of the recombinant protein; and/or aid in the purification of the recombinant protein by acting as a ligand in affinity purification.

Generally, the recombinant vectors also comprise sequences encoding an epitope tag (e.g. in the case of a prey protein) or a detectable substance (e.g. in the case of a bait protein) which facilitates the selection of the proteins. Suitable epitope tags and detectable substances are described herein.

An expressable recombinant vector may comprise a sequence encoding a protein that is an intracellular or extracellular signal (e.g. hormone, cytoplasmic signalling protein). An expressable recombinant vector may also comprise a sequence encoding a protein required for inducing signal transduction. For example, a vector may comprise a sequence of a TGFβ receptor that is required for TGFβ signalling.

In general, the recombinant vectors are expressable in host cells i.e. they are capable of replication in the host cell. It may be a DNA that is integrated into the host genome, and replicated as a part of the chromosomal DNA, or it may be a DNA which replicates autonomously, as in the case of a plasmid. In the latter case, the vector will include an origin of replication that is functional in the host. An integrating vector may include sequences which facilitate integration, e.g., sequences homologous to host sequences, or encoding integrases.

Recombinant vectors are introduced into host cells to produce recombinant cells. Recombinant cells include host cells which have been transformed or transfected with a recombinant expression vector. The terms “transformed with”, “transfected with”, “transformation” and “transfection” are intended to include the introduction of nucleic acid (e.g. a vector) into a cell by one of many techniques known in the art. Nucleic acid can be introduced into mammalian cells using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Suitable host cells for generating the recombinant cells and the methods and systems of the invention include a wide variety of eukaryotic host cells, preferably higher eukaryotic cells. Preferably, the host cells are mammalian cells. Examples of mammalian host cell lines include the COS-7 line of monkey kidney cells (ATCC CRL 1651) (Gluzman (1981) Cell 23:175) CV-1 cells (ATCC CCL 70), L cells, C127, 3T3, Chinese hamster ovary (CHO), HeLa and BHK cell lines. Other suitable host cells can be found in Goeddel, Gene Expression Technology: Methods in Enzymology 185, Academic Press, San Diego, Calif. (1991).

The recombinant cells may be engineered to produce a test agent/compound (e.g. drug).

Libraries

The invention provides a library comprising a mixture of nucleic acids comprising sequences encoding a variegated population of prey proteins involved in signal transduction pathways or cell cycle pathways. A library of the invention comprises nucleic acids encoding a large number of different potential proteins that comprise domains involved in signal transduction pathways. A library of the invention preferably comprises a set of nucleic acids encoding a variegated population of prey proteins and an epitope tag.

In an embodiment, the library is produced from a cDNA library.

In another embodiment, the library is derived to express a combinatorial library of proteins with known or unknown function that comprise a domain involved in signal transduction (e.g. SH2, SH3, PTB domain etc.). In preferred embodiments, the combinatorial polypeptides are in the range of at least 5, 10, 15, 20 or 25 amino acid residues in length. It will be understood that the length of the proteins does not reflect any extraneous sequences which may be present in order to facilitate expression, e.g., such as signal sequences or invariant portions of a fusion protein.

In a further embodiment, the library is derived to express a combinatorial library of polypeptides which are derived by mutagenesis of a known sequence. [See, for example, Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461]. Accordingly, polypeptide(s) which are known ligands for a target signaling molecule can be mutagenized by standard techniques to derive a variegated library of polypeptide sequences which can further be screened for agonists and/or antagonists.

An example of a library of the invention is the tagged signal transduction cDNA set described herein. In another embodiment, the library comprises nucleic acid molecules encoding cell cycle-related proteins.

A library may be prepared by introducing nucleic acids encoding the different proteins, in expressible form, into suitable host cells. The library may take the the form of a cell culture, in which essentially each cell expresses one, and usually only one, protein of the library. While the diversity of the library is maximized if each cell produces a protein of a different sequence, the library may have some redundancy. Depending on size, the proteins of the library can be expressed as is, or can be incorporated into larger fusion proteins. A fusion protein may provide, for example, stability against degradation or denaturation, as well as a detection signal.

In an embodiment, proteins of a library of the invention are encoded by a mixture of DNA molecules of different sequence. Each protein-encoding DNA molecule is ligated with a vector DNA molecule and the resulting recombinant DNA molecule is introduced into a host cell.

A library of the invention may comprise about 1000-20,000, 1000 to 10,000, 1000 to 5000, 1000 to 2000, or 1000 proteins or nucleic acids encoding proteins.

Methods

A recombinant or reagent cell or mixture thereof, or library of the invention, may be used to identify protein-protein interactions, and modulators that affect such interactions. Protein-protein interactions that lead to cell behaviour or gene responses may be identified by the methods of the invention.

Therefore, the invention provides a system for assaying for protein-protein interactions, and agents that affect such interactions comprising recombinant cells or a mixture or recombinant cells, or a library of the invention.

The invention relates to a method for identifying protein-protein interactions comprising prey proteins interacting with bait proteins comprising:
(a) introducing one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting detection of protein-protein interactions comprising a prey protein and the bait protein; and
(c) assaying for protein-protein interactions comprising a prey protein and bait protein by detecting the detectable substance.

In an aspect, the invention provides a method for identifying prey proteins that interact with one or more bait protein comprising:
(a) introducing one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting detection of protein-protein interactions comprising a prey protein and bait protein; and
(c) assaying for protein-protein interactions comprising a prey protein and bait protein by detecting the detectable substance.

In an embodiment, the present invention provides a method for identifying prey proteins that interact with one or more bait protein comprising:
(a) introducing one or more prey protein in cells, wherein a prey protein is labelled with an epitope tag permitting separation of the prey protein from other proteins in the cells;
(b) introducing one or more bait protein in the cells wherein a bait protein is labelled with a detectable substance permitting detection of the bait protein and protein-protein interactions comprising a bait protein and prey protein;
(c) inducing formation of protein-protein interactions between a prey protein and bait protein; and
(d) assaying for protein-protein interactions comprising a prey protein and bait protein.

The invention relates to a method for quantitating protein-protein interactions comprising a prey protein and a bait protein which method comprises the steps of:
(a) introducing one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting identification of the bait protein and protein-protein interactions comprising the bait protein and a prey protein;
(c) inducing formation of protein-protein interactions between a prey protein and bait protein; and
(d) quantitating protein-protein interactions comprising a prey protein and bait protein.

In an embodiment, a method for quantitating protein-protein interactions is provided which method comprises the steps of:
(a) expressing one or more prey protein in cells, wherein a prey protein is labelled with an epitope tag permitting separation of the prey protein from other proteins in the cells;
(b) expressing one or more bait protein in the cells wherein a bait protein is labelled with a detectable substance permitting identification of a bait protein and protein-protein interactions comprising the bait protein and a prey protein;
(c) obtaining a lysate of the cells and assaying an aliquot of the lysate to measure total expression of the epitope tag and detectable substance;
(d) assaying a second aliquot of the lysate to measure the amount of a detectable substance that coprecipitates with an epitope tagged prey protein;
(e) comparing the amounts measured in steps. (c) and (d) to quantitate the protein-protein interaction.

In a particular embodiment, the cells are subjected to an extracellular or intracellular signal after step (b). In a particular embodiment, FLAG is used as an epitope tag for one or more prey proteins and luciferase is used as a detectable substance for a bait protein.

The invention relates to a method for determining an interactome for one or more bait protein comprising:
(a) preparing recombinant cells each expressing one or more bait protein, in particular one bait protein, and one or mote prey protein selected from a variegated population of prey proteins;
(b) inducing formation of protein-protein interactions between prey proteins and bait proteins in the cells;
(c) identifying protein-protein interactions comprising a prey protein and bait protein to thereby determine the interactome.

In an embodiment, the bait protein is an unknown protein.

The invention also relates to a method for determining the function of a gene product comprising:
(a) defining an interactome of the gene product by preparing recombinant cells expressing the gene product and one or more prey protein selected from a variegated population of prey proteins, and identifying protein-protein interactions comprising the gene product and a prey protein; and
(b) determining the function of the gene product based on the structure and/or function of prey proteins that interact with the gene product in the interactome.

In a particular aspect, protein-protein interactions are directly identified, and in particular quantitated, using a mammalian cell system. A mammalian cell system provides upstream and downstream signaling apparatus of a specific protein-protein interaction. Thus, such a system may include receptors, protein trafficking events, subcellular localization and post-translational modifications.

The methods of the invention may further comprise a clustering step to identify protein-protein interactions that have similar dynamics and/or behaviour and thus may function as a coordinated response.

The methods of the invention can be designed to identify genes/proteins that physically interact with a protein/drug complex. For example, a bait protein may be complexed with a drug to identify prey proteins that interact with the complex. If the bait and prey proteins are able to interact in a drug-dependent manner, the interaction may be detected by detecting the detectable substance.

In an aspect the invention provides a method for preparing a profile of protein-protein interactions for a patient comprising:
(a) contacting recombinant cells expressing one or more prey protein selected from a variegated population of prey proteins with proteins isolated from the patient;
(b) identifying protein-protein interactions between prey proteins and proteins isolated from the patient;
(c) preparing a profile of the identified protein-protein interactions.

In an embodiment, the patient has or is suspected of exhibiting a disease or condition associated with the isolated proteins or interactions comprising the isolated proteins. In another embodiment, the patient profile is compared with a profile generated for a standard. The standard may be a normal subject or a subject with a different disease stage. A patient profile may also be compared with a profile prepared for the same patient at a different time (e.g. after therapy or surgery, or at a different disease stage).

The methods of the invention permit the identification and analysis of changes in active proteins in different cell types and under different conditions, and are useful in addressing the biochemical mechanisms of disease.

In an aspect the invention provides a method for systematically analyzing protein-protein interactions in cell signalling comprising:
(a) introducing into mammalian cells (i) one or more prey protein labeled with an epitope tag permitting separation of the prey protein from other proteins in the cells; and (ii) one or more bait protein labelled with a detectable substance permitting identification of the bait protein and protein-protein interactions comprising the bait protein and a prey protein;
(b) inducing cell signaling in the cells to thereby form protein-protein interactions between a prey protein and bait protein;
(c) assaying for protein-protein interactions comprising a prey protein and bait protein at different time points; and
(d) comparing the types of protein-protein interactions at the different time points.

A method for quantitatively analyzing protein-protein interactions in cell signalling comprising:
(a) introducing into mammalian cells (i) one or more prey protein labeled with an epitope tag permitting separation of the prey protein from other proteins in the cells; and (ii) one or more bait protein labelled with a detectable substance permitting identification of the bait protein and protein-protein interactions comprising the bait protein and a prey protein;
(b) inducing cell signaling in the cells to thereby form protein-protein interactions comprising a prey protein and bait protein; and
(c) quantitating protein-protein interactions comprising a prey protein and bait protein at different time points.

The invention also provides a method for determining changes in an interactome of a mitotic kinase during cell cycle progression comprising:
(a) introducing into mammalian cells (i) one or more prey protein labeled with an epitope tag permitting separation of the prey protein from other proteins in the cells; and (ii) one or more mitotic kinase labelled with a detectable substance permitting identification of the mitotic kinase and protein-protein interactions comprising the mitotic kinase and a prey protein;
(b) assaying for protein-protein interactions comprising a prey protein and mitotic kinase at different time points; and
(c) comparing the types and kind of protein-protein interactions at the different time points.

In an embodiment the mitotic kinase is a serine/threonine kinase.

A method for analyzing protein-protein interactions in different cell types comprising:
(a) introducing into first cells, in particular mammalian cells, (i) one or more prey protein labeled with an epitope tag permitting separation of the prey protein from other proteins in the cells; and (ii) one or more bait protein labelled with a detectable substance permitting identification of a bait protein and protein-protein interactions comprising the bait protein and a prey protein;
(b) introducing into second cells, in particular mammalian cells, the same prey protein(s) and bait protein(s) introduced into the first cells in step (a);
(c) inducing cell signalling in the cells in (a) and (b) to thereby form in the first and second cells protein-protein interactions comprising a prey protein and bait protein; and
(d) comparing the protein-protein interactions identified in the first cells with the protein-protein interactions in the second cells.

In an embodiment, the first cells are from a subject with a disease and the second cells are normal cells.

The invention provides a method for assaying for changes in protein-protein interactions in response to intracellular or extracellular factors comprising:
(a) introducing one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting identification of the bait protein and protein-protein interactions comprising the bait protein and a prey protein;
(c) inducing formation of protein-protein interactions between a prey protein and bait protein;
(d) introducing an intracellular or extracellular factor;
(e) assaying protein-protein interactions comprising a prey protein and bait protein; and
(f) comparing the assayed protein-protein interactions with protein-protein interactions assayed in the absence of the intracellular or extracellular factor.

The invention permits the identification of agents or compounds that interact with and modulate the activity of a protein-protein interaction or component thereof and are potentially useful as therapeutics. Thus, the present invention provides a convenient format for discovering drugs that can be useful to modulate cellular function, as well as to understand the pharmacology of agents or compounds that specifically modulate protein-protein interactions.

In an aspect the invention provides a method for evaluating a compound for its ability to modulate a signal transduction pathway through a prey protein, bait protein, or protein-protein interaction of the invention. For example, the compound may be a substance which binds to a prey protein or protein-protein interaction, or which disrupts or promotes the interaction of proteins in a protein-protein interaction.

The invention also provides a method for identifying an agent to be tested for an ability to modulate a signal transduction pathway by testing for the ability of the agent to affect the interaction between molecules in a protein-protein interaction, wherein the protein-protein interaction is part of the signal transduction pathway.

In an embodiment the invention provides a method for identifying a potential modulator of signal transduction activity comprising:
(a) introducing one or more prey protein in mammalian cells, wherein a prey protein is labelled with an epitope tag permitting separation of the prey protein from other proteins in the cells;
(b) introducing one or more bait protein in the cell wherein a bait protein is labelled with a detectable substance permitting identification of the bait protein and protein-protein interactions comprising the bait protein and a prey protein;
(c) introducing a test agent in the cell;
(d) inducing formation of protein-protein interactions between a prey protein and bait protein;
(e) assaying protein-protein interactions comprising the prey protein and bait protein; and
(f) comparing the protein-protein interactions with the protein-protein interactions obtained in the absence of the test agent to determine the effect of the test agent on the protein-protein interactions wherein a change in the protein-protein interactions indicates that the test agent is a potential modulator.

In an embodiment, the invention relates to a method for screening for an agent or compound that affects a protein-protein interaction comprising:
(a) introducing one or more prey protein in mammalian cells, wherein the prey protein is labelled with an epitope tag permitting separation of the prey protein from other proteins in the cells;
(b) introducing one or more bait protein in the cells, wherein the bait protein is labelled with a detectable substance permitting identification of the bait protein and protein-protein interactions comprising the bait protein and a prey protein;
(c) introducing a test agent in the cells;
(d) inducing formation of protein-protein interactions between the prey protein and bait protein;
(e) assaying protein-protein interactions comprising the prey protein and bait protein; and
(f) comparing the protein-protein interactions with the protein-protein interactions obtained in the absence of the test agent to determine the effect of the test agent on the protein-protein interactions wherein an increase in the protein-protein interactions indicates that the agent is an agonist of the interaction and a decrease in the amount of protein-protein interactions indicates that the agent is an antagonist.

In another embodiment, the invention provides a method for identifying inhibitors of an interaction between a prey protein and a bait protein, comprising:
(a) introducing one or more prey protein in cells, wherein the prey protein is labelled with an epitope tag permitting separation of the prey protein from other proteins in the cells;
(b) introducing one or more bait protein in the cell wherein the bait protein is labelled with a detectable substance permitting identification of the bait protein and protein-protein interactions comprising the bait protein and prey protein;
(c) introducing a test agent in the cells;
(d) inducing formation of protein-protein interactions between a prey protein and bait protein;
(e) assaying protein-protein interactions comprising a prey protein and bait protein; and
(f) comparing the protein-protein interactions with the protein-protein interactions obtained in the absence of the test agent to determine the effect of the agent on the protein-protein interactions, wherein a decrease in the amount of protein-protein interactions indicates that the agent is an inhibitor.

Generally the conditions for inducing formation of protein-protein interactions in the methods of the invention may be selected having regard to factors such as the nature and amounts of the prey and bait proteins, and optionally the test agent. The interaction between a prey protein and bait protein may be induced by introducing an intracellular or extracellular signal. The interaction may also be promoted or enhanced either by increasing production of one of the proteins, by increasing expression of one of the proteins, or by promoting interaction of the proteins by prolonging the duration of the interaction.

Protein-protein interactions may be isolated by conventional isolation techniques, for example, salting out, chromatography, electrophoresis, gel filtration, fractionation, absorption, polyacrylamide gel electrophoresis, agglutination, or combinations thereof. To facilitate the isolation of the protein-protein interactions, the prey protein is labelled with an eptitope tag and the bait protein is labelled with a detectable substance.

In an embodiment, the protein-protein interactions are isolated by purifying the epitope tagged prey protein and complexes comprising the epitope tagged prey protein (e.g. using magnetic affinity resins coated with anti-epitope antibody), and co-purifying protein-protein interactions comprising the epitope tagged prey protein and the labeled bait protein by detecting the detectable substance (e.g. enzymatic detection).

The methods may be carried out in the liquid phase or the proteins, or test compound may be immobilized. One or more of a prey protein, bait protein, and/or test agent, preferably the prey protein used in a method of the invention may be insolubilized. For example, a protein may be directly or indirectly (e.g. with an antibody) bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or agent may be prepared by reacting the material with a suitable insoluble carrier using known chemical or physical methods, for example, cyanogen bromide coupling.

Still another aspect of the present invention provides a method of conducting a drug discovery business comprising:
(a) providing one or more methods or assay systems of the invention for identifying agents by their ability to inhibit or potentiate a protein-protein interaction;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:
(a) providing one or more methods or assay systems of the invention for identifying agents by their ability to inhibit or potentiate a protein-protein interaction;
(b) (optionally) conducting therapeutic profiling of agents identified in step (a) for efficacy and toxicity in animals; and
(c) licensing, to a third party, the rights for further drug development and/or sales for agents identified in step (a), or analogs thereof.

The methods of the invention may also be used generally to detect mutations in cellular proteins that disrupt protein-protein interactions. Mutations in genes encoding either a bait or prey protein which result in disruption of the interaction between the bait and prey protein can be detected using the methods of the invention.

Thus, the methods of the invention can be used to map residues of a protein involved in a known protein-protein interaction. Thus, various forms of mutagenesis can be utilized to generate a library of either bait or prey proteins, and the ability of the mutant proteins to function in a method of the invention may be assayed. The methods of the invention can be used to identify mutations that result in diminished binding between bait proteins and prey proteins.

The methods of the invention can be used in the form of a diagnostic assay to detect the interaction of two proteins, for example, where the protein or gene encoding same is isolated from biopsied cells. The methods of the invention may be used to detect mutants which while expressed at appreciable levels in the cell are defective at binding other cellular proteins. Mutants may arise from point mutations that may be impractical to detect by diagnostic sequencing techniques or immunoassays.

The present invention thus contemplates a diagnostic screening assay to detect the presence of a mutant of a bait protein or gene encoding a bait protein in cells from a sample comprising;
(a) cloning cDNAs from the cells which encode a bait protein or a mutant thereof;
(b) expressing in a host cell the cloned cDNAs and one or more prey protein under conditions which permit the detection of an interaction between the bait protein and a prey protein, wherein a prey protein is labelled with an epitope tag permitting separation of a prey protein from other proteins in the cell, and the bait protein is labelled with a detectable substance permitting identification of the bait protein and protein-protein interactions comprising the bait protein and prey protein; and
(c) detecting protein-protein interactions wherein a decrease in protein-protein interactions compared to a control using a normal bait protein indicates that the bait protein or gene encoding a bait protein in the cells is potentially mutated.

In an aspect of the invention, a method is provided for constructing a protein linkage map for a proteome or interactome comprising:
(a) identifying interactions between proteins in a variegated protein library and a selected set of bait proteins from the proteome, in the presence or absence of extracellular or intracellular factors; and
(b) displaying the interactions as a protein linkage map.

The invention provides a matrix comprising a color gradient displaying the magnitude of one or more protein-protein interactions identified using a method of the invention. In an embodiment, the matrix is a series of similar colored shapes (e.g. squares) each shape representing the interaction density of a protein-protein interaction. In an aspect, the matrix comprises 100 by 100, or 10,000 individual protein-protein interactions.

The invention provides libraries of information on protein-protein interactions, efficient methods to construct such libraries, and data sharing systems which enable efficient utilization of such libraries. The invention also provides databases which accommodate and maintain libraries of information relative to protein-protein interactions identified in accordance with the invention, methods and systems to construct the databases by accumulating those pieces of information which concern protein-protein interactions as they relate to various biological systems, methods and systems to enable a client to search through the databases for desired information, methods and systems to transmit to the client desired pieces of information concerning protein-protein interactions that are housed in the databases, tangible electronic means to record and make use of the systems and databases, and apparatus to enable construction and search of the data bases and/or transmission of desired information to a client.

Therefore, methods of the invention may further comprise inputing and analyzing data on protein-protein interactions from the methods described herein in a computerized system.

In an aspect the invention provides a database of interacting proteins. Information produced by the methods of the invention can be stored on a computer readable medium. Therefore, the invention provides a computer readable medium or a machine readable storage medium which comprises protein-protein interactions or interactomes identified using a method of the invention. Such storage medium or storage medium encoded with these data are capable of displaying on a computer screen or similar viewing device, a representation of such interactions or interactome. Thus, the invention also provides computerized representations of protein-protein interactions or interactomes identified using a method of the invention, including any electronic, magnetic, or electromagnetic storage forms of the data needed to define the interactions or interactome such that the data will be computer readable for purposes of display and/or manipulation.

The invention also provides a computer for the analysis of protein-protein interactions or an interactome wherein the computer comprises:
(a) a machine-readable data storage medium comprising a data storage material encoded with machine readable data wherein the data comprises protein-protein interactions or an interactome characterized using a method of the invention;
(b) a working memory for storing instructions for processing said machine-readable data of (a);
(c) a central-processing unit coupled to the working memory and to the machine-readable data storage medium of (a) for performing a Fourier transform of the machine readable data of (a) and for processing the machine readable data of (b) into protein-protein interactions and interactome; and
(d) a display coupled to the central-processing unit for displaying the protein-protein interactions and interactome.
   High Throughput/Robotics Systems

The methods of the invention can be carried out in a high throughput format. In particular, in drug screening programs that test libraries of proteins, compounds, and natural extracts, high throughput assays are desirable in order to maximize the number of compounds screened in a given period of time.

The methods of the invention may be used in robotics systems that can handle large numbers of samples for proportioning, mixing, and sample-handling. The invention therefore makes available robotics that can perform multiple reactions at variable temperatures, and subsequently handle work up and characterization of protein-protein interactions, and agents/compounds/modulators identified using a method of the invention Robotics systems for implementing the methods of the invention may utilize simple, automated dilution devices or the systems may be highly evolved workstations in which multiple functions are performed by one or more mechanical arms. In the preferred embodiment of the invention, full automation (i.e., from sample dispensing to data collection) allows for round-the-clock operation, thereby increasing the overall screening rate and mitigating the potential for human error common in highly redundant procedures. Examples of suitable robotic systems or components of same for implementing the methods of the invention are described in U.S. Pat. No. 6,253,807, or are commercially available from Thermo-CRS (Burlington, Ontario, Canada) or Beckman Coulter (Calif., U.S.).

The identification of protein-protein interactions and active compounds within libraries using the methods described herein can be followed or confirmed by other identification procedures. For example, x-ray crystallographic studies may be used as a means of evaluating protein-protein interactions. Purified recombinant molecules when crystallized in a suitable form are amenable to detection of intra-molecular interactions by x-ray crystallography. Mass spectroscopy may also be used to detect interactions and in particular, Q-TOF instrumentation may be used. Two-hybrid systems may also be used to detect protein interactions.

The invention also provides an integrated modular system for performing the methods of the invention. In an embodiment, the system comprises one or more of the following modules:
(a) a culture system module comprising microtiter plate wells containing recombinant cells of the invention;
(b) a module for retrieving cDNA clones encoding prey proteins or test agents;
(c) an automated immunoprecipitation module for affinity purification of proteins of a protein-protein interaction or test agents;
(d) an analysis module for further purifying the proteins or agents from (c) or preparing fragments of such proteins or agents that are suitable for mass spectrometry;
(e) a mass spectrometer module for automated analysis of fragments from (d);
(f) a computer module comprising an integration software for communication among the modules of the system and integrating operations;
(g) a module for retrieving cDNA clones encoding prey proteins or test agents; and
(h) a module for performing an automated method of the invention.
   Kits

The methods of the present invention, as described above, may be practiced using kits for detecting and characterizing interactions between bait proteins and prey proteins. A kit will generally include expressable recombinant vectors for generating one or more bait protein labelled with a detectable substance and for generating one or more prey protein labelled with an eptiope tag, and a host cell. Binding of a bait protein and a prey protein in a host cell results in measurable change in expression of a detectable substance, e.g., relative to the absence of an interaction between the two proteins.

In certain embodiments, one or both of the expression vectors can be integrated into the genome of the host cell. The first vector contains a promoter and other relevant transcription and/or translation sequences to direct expression of one or more prey protein nucleic acid. Also included on the first vector is one or more epitope tag, which in the host cell permits selection of cells containing a prey protein. The second vector is derived for generating one or more bait protein. A bait protein gene includes a promoter and other relevant transcription and/or translation sequences to direct expression of one or more bait protein gene. The second vector also includes one or more detectable substance nucleic acid, the expression of which in the host cell permits selection of cells containing a bait protein or protein-protein interactions comprising a bait protein and a prey protein.

The kit includes a host cell, preferably a mammalian cell, which can be engineered to express the bait and prey proteins, and express the detectable substance(s) in a manner dependent on the formation of protein-protein interactions including the bait and prey proteins. The host cell, by itself, is preferably incapable of expressing a protein having a function of a prey protein or a bait protein.

Accordingly, in using the kit the interaction of bait and prey components of the proteins in the host cell causes a measurable change in expression of a detectable substance relative to the case where the test proteins do not interact. The detectable substance gene may encode an enzyme or other product that can be readily measured. In an embodiment a detectable substance gene encodes Renilla luciferase. Such measurable activity may include the presence of detectable enzyme activity only when the gene is transcribed.

Agents/Compounds

The methods described herein are designed to screen or identify agents/compounds that modulate a protein-protein interaction thus affecting signal transduction activity or pathways. Agents/compounds are therefore contemplated that interact with or bind to a protein-protein interaction or component thereof, or bind to other proteins that interact with the interaction or component thereof, to compounds that interfere with, or enhance the interaction of molecules in a protein-protein interaction. The methods of the invention may also be used generally to detect mutations in cellular proteins that disrupt protein-protein interactions.

The agents/compounds identified using the methods of the invention include but are not limited to peptides such as soluble peptides including Ig-tailed fusion peptides, members of protein or peptide libraries and combinatorial chemistry-derived molecular libraries made of D- and/or L-configuration amino acids, polysaccharides, oligosaccharides, monosaccharides, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agent/compound may be an endogenous physiological compound or it may be a natural or synthetic compound.

Lead compounds may be identified for drug development. The structure of the compounds can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified compounds, or other small molecules or lead compounds that can be tested for predicted properties as related to the target molecule. The activity of the lead compounds can be evaluated using standard in vitro and in vivo procedures appropriate for the target.

Information about structure-activity relationships may also be obtained from co-crystallization studies. In these studies, an agent with a desired activity is crystallized in association with a target molecule, and the X-ray structure of the complex is determined. The structure can then be compared to the structure of the target molecule in its native state, and information from such a comparison may be used to design compounds expected to possess desired activities.

A lead compound may be used to design small molecule mimetics, agonists, or antagonists. A drug design method may involve determining the three dimensional structure of the compound and providing a small molecule or peptide capable of binding to a ligand binding site on the compound. Those skilled in the art will be able to produce small molecules or peptides that mimic the effect of the compound partner and that are capable of easily entering the cell. Once a molecule is identified, the molecule can be assayed for its ability to bind a protein of a protein-protein interaction (e.g. using a method of the invention), and the strength of the interaction may be optimized by making amino acid deletions, additions, or substitutions of by adding, deleting, or substituting a functional group. The additions, deletions, or modifications can be made at random or may be based on knowledge of the size, shape, and three-dimensional structure of the compound.

Computer modelling techniques known in the art may also be used to observe the interaction of a compound with a protein of a protein-protein interaction (for example, Homology Insight II and Discovery available from BioSym/Molecular Simulations, San Diego, Calif., U.S.A.). If computer modelling indicates a strong interaction, a compound can be synthesized and tested for its ability to interfere with the binding of a protein with an interacting molecule.

Secondary assays and animal models can also be used to identify lead compounds and/or confirm the activity of an agent identified using a method of the invention. For example, agents/compounds that affect the interaction of protein-protein interactions in the TGFβ signaling transduction pathway may be tested to determine if they affect TGF-β1-dependent regulation of cell proliferation and gene responses. Compounds/agenst may be tested in Mink lung (Mv1Lu) and in TGF-β-responsive human cells (HepG2) for relief of growth inhibitory and gene responses to TGF-β1.

Proteomics Analyses

Proteomics research is a critical component of functional genomics. The methods described herein, in particular the high throughput technologies, allow for rapid cell-based analysis of protein-protein interactions in mammalian systems. Application of this technology to a quantitative analysis of signal transduction interactomes will provide novel insights into how biological responses are controlled in complex systems and how molecular alterations in signalling pathways manifest themselves as disease. In addition the present invention provides critical resources in the nascent field of modelling biological systems.

Application of high throughput technology is a key element to permit proteomics analyses on a genome-wide scale. Protein-protein interactions that are critical for disease progression can be directly targeted for drug discovery in a cell-based assay. Furthermore, development of efficient high throughput assays of protein-protein networks can be utilized in rapid profiling of drug candidates to evaluate specificity.

The invention may have particular application in the analysis of protein-protein interactions involved in receptor tyrosine kinase (RTK) pathways. These pathways regulate many of the activities of animal cells and aberrant functions contribute to a variety of human cancers. The most extensively studied signalling pathways are those mediated by the transmembrane receptor tyrosine kinases (RTK) (Hunter, T. (2000). Cell , 100, 113-127; Pawson, T., and Saxton, T. M. (1999). Cell 97(97), 675-678; and Pawson, T., and Nash, P. (2000). Genes Dev. 14, 1027-1047). RTKs are important regulators of development and cell communication and perturbation of RTK signalling results in malignant transformation. Following activation by ligand binding, RTKs, such as the PDGF receptor undergo autophosphorylation at Tyr residues which can bind cytoplasmic targets with phosphotyrosine (pTyr) recognition modules, namely SH2 and PTB domains. These receptor interacting proteins can be enzymes (such as phospholipase C), adapters that physically link the receptor to an enzyme (such as Grb2 which recruits the Ras nucleotide exchange factor SOS), latent transcription factors (such as STATs), scaffolding proteins (such as Shc) or negative regulators (such as Cbl). While in some cases, alterations in gene expression can occur in a fairly direct manner as for the DNA-binding STATs, most RTKs signal through diverse pathways that include phospholipid kinases, phospholipases, small GTPases and cascades of protein kinases that include. MAP kinases such as Erk and Ink Studies of these signalling effectors indicate that these proteins form networks of interactions rather than simple linear pathways. Signalling specificity is thought to be achieved partially through binding of specific SH2-domain containing proteins, each of which can preferentially bind to distinct pTyr motifs. However, accumulating evidence indicates that distinct SH2-domain containing proteins can function redundantly in signal transduction. Thus, a current challenge in the field is to elucidate how specific cellular responses are achieved when many of the same core pathways are activated by different receptors. One possibility is that a cell might convert differences in amplitude and duration of pathway activation into qualitatively different biological responses. Thus, understanding the RTK signal transduction interactome in time and in response to activation of different receptors is of great interest and has applications for the treatment and prevention of diseases such as cancer.

Compositions and Treatments

The agents/compounds identified using the methods of the invention may be formulated into compositions for administration to individuals suffering from a disease or condition. Therefore, the present invention also relates to a composition comprising one or more of an agent/compound identified using a method of the invention, and a pharmaceutically acceptable carrier, excipient or diluent. A method for modulating a signal transduction activity associated with a disease or condition is also provided comprising introducing into the cells an agent/compound identified using a method of the invention or a composition containing same.

An agent or compound identified using the methods of the invention may be used to modulate signal transduction pathways that control cellular processes such as proliferation, growth, and/or differentiation of cells.

Thus, the agents/compounds identified using the methods of the invention may be formulated into compositions for administration to individuals suffering from a proliferative or differentiative condition. Therefore, the present invention also relates to a composition comprising an agent or compound, and a pharmaceutically acceptable carrier, excipient or diluent. A method for modulating proliferation, growth, and/or differentiation of cells is also provided comprising introducing into the cells an agent or compound that inhibits a protein-protein interaction associated with cell proliferation, growth, and/or differentiation, or a composition containing same.

Still further the invention provides the use of agent/compound identified using a method of the invention in the preparation of a medicament to treat individuals suffering from a disease or condition.

In an embodiment, the invention provides the use of an agent in the preparation of a medicament to modulate cell prolilferation, growth, and/or differentiation in cells of an individual. The invention also contemplates the use of an agent in the preparation of medicament to treat individuals suffering from a proliferative or differentiative condition.

The disruption or promotion of the interaction between the molecules in protein-protein interactions is also useful in therapeutic procedures. Therefore, the invention features a method for treating a subject having a condition characterized by an abnormality in a signal transduction pathway involving a protein-protein interaction. The abnormality may be characterized by an abnormal level of interaction between the interacting molecules. An abnormality may be characterized by an excess amount, intensity, or duration of signal or a deficient amount, intensity, or duration of signal. An abnormality in signal transduction may be realized as an abnormality in cell function, viability, or differentiation state. The method involves disrupting or promoting the interaction (or signal) in vivo, or the activity of the protein-protein interaction. A compound that will be useful for treating a disease or condition characterized by an abnormality in a signal transduction pathway involving a protein-protein interaction can be identified by testing, using a method of the invention, the ability of the compound to affect (i.e. disrupt or promote) the interaction between the molecules in the protein-protein interaction. The compound may promote the interaction by increasing the production, or by increasing expression of a protein of a protein-protein interaction, or by promoting the interaction of the molecules. The compound may disrupt the interaction by reducing the production of a protein, preventing expression of a protein, or by specifically preventing interaction of the molecules in the complex.

In yet another aspect the invention provides a method of treating diseases or conditions where the affected cells have a defective prey or bait protein (e.g. mutated target protein or over expressed target protein) comprising administering an effective amount of an agent or compound identified using a method of the invention.

An agent or compound herein can be administered to a subject either by themselves, or they can be formulated into pharmaceutical compositions for administration to subjects in a biologically compatible form suitable for administration in vivo. By “biologically compatible form suitable for administration ill vivo” is meant a form of the agent/compound to be administered in which any toxic effects are outweighed by the therapeutic effects.

The agents/compounds may be administered to living organisms including humans, and animals (e.g. dogs, cats, cows, sheep, horses, rabbits, and monkeys). Preferably the agents/compounds are administered to human and veterinary patients.

An agent/compound may be administered in a therapeutically active amount A “therapeutically active amount” is defined as an amount of a substance, at dosages and for periods of time necessary to achieve the desired result. For example, a therapeutically active amount of an agent/compound may vary according to factors such as the disease state, age, sex, and weight of the individual, and the ability of the agent/compound to elicit a desired response in the individual. Dosage regime may be adjusted to provide the optimum therapeutic response. For example, several divided doses may be administered daily or the dose may be proportionally reduced as indicated by the exigencies of the therapeutic situation. A therapeutically active amount can be estimated initially either in cell culture assays e.g. of neoplastic cells, or in animal models such as mice, rats, rabbits, dogs, or pigs. Animal models may be used to determine the appropriate concentration range and route of administration for administration to humans.

The active substance may be administered in a convenient manner by any of a number of routes including but not limited to oral, subcutaneous, intravenous, intraperitoneal, intranasal, enteral, topical, sublingual, intramuscular, intra-arterial, intramedullary, intrathecal, inhalation, transdermal, or rectal means. The active substance may also be administered to cells in ex vivo treatment protocols. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the agents or compounds in association with one or more pharmaceutically acceptable vehicles or diluents, and contained in buffered solutions with a suitable pH and iso-osmotic with the physiological fluids.

An agent or compound can be in a composition which aids in delivery into the cytosol of a cell. The substance may be conjugated with a carrier moiety such as a liposome that is capable of delivering the substance into the cytosol of a cell (See for example Amselem et al., Chem. Phys. Lipids 64:219-237, 1993 which is incorporated by reference). Alternatively, an agent or compound may be modified to include specific transit peptides or fused to such transit peptides that are capable of delivering the substance into a cell. The agents or compounds can also be delivered directly into a cell by microinjection.

An agent or compound may be therapeutically administered by implanting into a subject, vectors or cells capable of producing the agent or compound. In one approach cells that secrete an agent or compound may be encapsulated into semipermeable membranes for implantation into a subject The cells can be cells that have been engineered to express an agent or compound. It is preferred that the cell be of human origin.

A nucleic acid encoding an agent or compound may be used for therapeutic purposes. Viral gene delivery systems may be derived from retroviruses, adenoviruses, herpes or vaccinia viruses or from various bacterial plasmids for delivery of nucleic acid sequences to the target organ, tissue, or cells. Vectors that express the agent or compound can be constructed using techniques well known to those skilled in the art (see for example, Sambrook et al.). Non-viral methods can also be used to cause expression of an agent or compound in tissues or cells of a subject Most non-viral methods of gene transfer rely on normal mechanisms used by mammalian cells for the uptake and transport of macromolecules. Examples of non-viral delivery methods include liposomal derived systems, poly-lysine conjugates, and artificial viral envelopes.

In viral delivery methods, vectors may be administered to a subject by injection, e.g. intravascularly or intramuscularly, by inhalation, or other parenteral modes. Non-viral delivery methods include administration of the nucleic acids using complexes with liposomes or by injection; a catheter or biolistics may also be used.

The activity of an agent, compound, or compositions of the invention may be confirmed in animal experimental model systems. The therapeutic efficacy and safety of an agent, compound, or composition can be determined by standard pharmaceutical procedures in cell cultures or animal models. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD50 ratio. Pharmaceutical compositions which exhibit large therapeutic indices are preferred.

By way of example, agents/compounds that modulate the TGFβ pathway can be assessed in mice for suppression of wound-induced fibrosis in skin (Shah M et al, J. Cell Science 108:985-1002, 1995), and subsequently for suppression of BCG-induced lung fibrosis and inflammation (Denis M. Immunlogy 82:584-590, 1994). Neutralizing anti-TGF-β1 antibodies can be used as a positive control in the therapy experiments, to verify that blocking TGF-β is indeed therapeutically effective. Affected tissues can be monitored for collagen matrix deposition, inflammatory cytokines transcripts by RNase protection and proteins by ELISA assays.

Antibodies that specifically bind a therapeutically active ingredient may be used to measure the amount of the therapeutic active ingredient in a sample taken from a patient for the purposes of monitoring the course of therapy.

The invention also contemplates a method for evaluating a condition or disease of a patient suspected of exhibiting a condition or disease involving a protein-protein interaction. For example, biological samples from patients suspected of exhibiting a disease or condition may be assayed for the presence of the interaction using a method of the invention. If a protein-protein interaction is normally present, and the development of the disease or condition is caused by an abnormal quantity of one or both proteins of the interaction, the assay should compare levels of the interaction in the biological sample to the range expected in normal tissue of the same type.

An interactome may be detemined for a patient and compared to a standard to identify differences between the protein-protein interactions in the interactome and the standard. Identification of differences may assist in the diagnosis, prognosis, or treatment of a disease or condition.

The following non-limiting example is illustrative of the present invention:

### EXAMPLE 1

Development of a High Throughput (HTP) Protein-Protein Interaction (PPI) Assay.

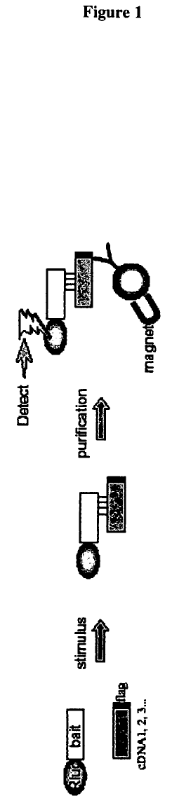

Defining the mammalian signal transduction interactome using in vitro, prokaryotic or yeast-based systems is of limited value, primarily because much of the signalling apparatus that exists upstream and downstream of a specific interacting protein pair is missing in these systems. Furthermore, key receptors, protein trafficking events, subcellular localization and posttranslational modifications may not be accurately recapitulated. Therefore, a method was devised to quantitate specific PPIs rapidly and directly in a stimulus-dependent manner using a mammalian cell-based system. This technology involves expressing an epitope-tagged version of protein A together with protein B that is engineered to contain a detection tag that requires SDS-PAGE-based analysis (FIG. 1). Protein B bound to protein A can then be directly measured in immunoprecipitates.

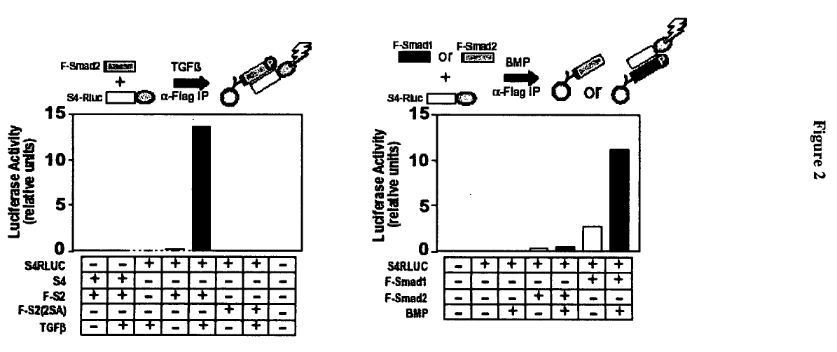

To develop the methodology the Smad pathway was used as a model system. R-Smad2 was tagged with the flag epitope tag, which allows for efficient recovery of tagged protein complexes from mammalian cell lysates. Next the Smad2 partner, Smad4, was tagged with a detection tag. Fluorescence-based detection of the fusion partner was tried but the sensitivity was not sufficiently high. Therefore, an enzymatic tag was selected. Luciferase enzymes were selected which provide one of the most sensitive enzymatic assays known. Firefly luciferase was inactive when fused to Smad4, however Renilla luciferase retained robust activity. Next the interaction of Flag-Smad2 with Smad4-Rluc was investigated (FIG. 2). For this, lysates from cells expressing the indicated cDNAs were immunoprecipitated using anti-flag antibody and the Smad2 bound to protein A-sepharose subjected to a Renilla luciferase assay to detect bound Smad4-Rluc. In the absence of either Smad2 expression or TGFβ signalling, little Smad4-Rluc was precipitated. In contrast, in the presence of TGFβ signalling, which induces phosphorylation of Smad2 and drives heteromeric complex formation with Smad4, a strong enhancement in Smad4-Rluc bound to wild type Smad2 was detected but not a phosphorylation site mutant of Smad2. Similar results were obtained when the binding of Smad4-Rluc with R-Smad1, which is activated by BMP but not TGFβ receptors, was assessed. Together, these data demonstrate that luciferase is a sensitive detection tag that can be utilized to detect protein-protein interactions in mammalian cell-based assays.

Modification of the PPI Assay into a HTP Format and Analysis of a Pilot Interactome Screen

The assays described above were conducted manually, however to map PPIs in a HTP screen requires automated, robotics-based technologies. To do this, the method was modified by shifting all cell culture and transfections into a 96-well format and developed HTP immunoprecipitation methods using magnetic bead technology.

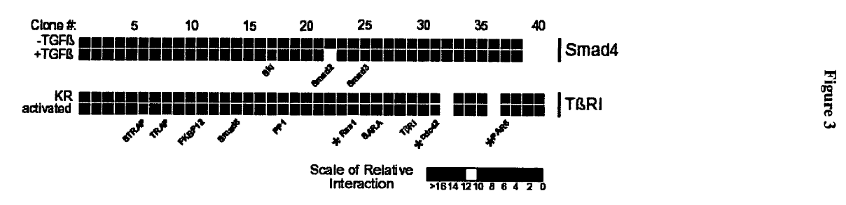

To conduct a pilot interactome screen a collection of 40 Flag-tagged cDNAs were assembled and automated liquid handling procedures were developed using a Packard Multiprobe robot. For bait, Smad4-Rluc, which is described above, was selected and the TGFβ type I receptor was fused at the C-terminus to Rluc. Smad4-Rluc was screened in the presence and absence of TGFβ signalling, while kinase-deficient inactive and a constitutively active version of TβRI were employed for the receptor screen. This pilot screen thus assessed approximately 160 interactions in triplicate so as to evaluate assay precision. To visualize the data each interaction test is presented as a box, with the relative magnitude of the interaction colour-coded (FIG. 3). In general variability was low and standard deviations of positive interactions were consistently in the range of 5-10%. In the Smad4 screen, three proteins were found to interact with Smad4-Rluc. One, the proto-oncogene Ski interacted with Smad4 both in the absence and presence of TGFβ signalling, as previously reported (Attisano, L., and Wrana, J. L. (2000). Curr. Op. Cell Biol. 12, 235-243; Wrana, J. L., and Attisano, L. (2000). Cyto. Growth Factor Rev. 11, 5-13; and Massagué, J., Blain, S. W., and Lo, R. S. (2000). Cell 103, 295-309). The other two proteins were R-Smad2 and R-Smad3, which showed a strong TGFβ-dependent interaction as described above. Of note, R-Smad1 did not interact with Smad4, consistent with its role in BMP but not TGFβ signalling pathways. Next the pilot-scale interactome of the TGFβ type I receptor was examined. As previously reported, TβRI bound strongly to itself and FKBP12 and more weakly to protein phosphatase I, STRAP and TRAP (Attisano, L., and Wrana, J. L. (2000). Curr. Op. Cell Biol. 12, 235-243; Wrana, J. L., and Attisano, L. (2000). Cyto. Growth Factor Rev. 11, 5-13; and Massagué, J., Blain, S. W., and Lo, R. S. (2000). Cell 103, 295-309). Interestingly, in the course of this screen some novel interactions were detected. One of these, the strong interaction between PAR6 and TβRI was of particular interest because cdc42, which also interacted with TβRI, is a partner of PAR6 (Kim, S. K. (2000). Nat. Cell Biol. 2, E143-145). This interaction was tested further and TGFβ receptor complexes affinity-labelled with ¹²⁵I-TGFβ were found to coprecipitate with PAR6 (data not shown).

These studies thus demonstrate the efficacy of applying this screen in a HTP format for the genome-wide analysis of signal transduction interactomes in mammalian cells. Further, they indicate that the assay can be applied to the analysis of transmembrane receptors and to the discovery and characterization of novel protein-protein interactions.

Development of an Automated Robotics Platform to Detect PPIs in Mammalian Cells.

To conduct the large scale screens that are required to build a genome-wide view of protein-protein interactions, two resources were utilized. The first is an integrated robotics platform developed with Thermo CRS. (Burlington, ON, Canada). The robot performs all tissue culture, liquid handling steps, magnetic bead purification and detection. The second resource is the tagged cDNA library. A full length cDNA library collection called the FANTOM set was obtained from RIKEN Genome Sciences Center (RIKEN GSC) in Japan. The set has about 20,000 full-length annotated mouse cDNA clones (20) and was be used for the efficient construction of a large number of tagged cDNAs.

Development of a Tagged Signal Transduction cDNA Set.

A library of modified cDNAs was developed using a customized topisomerase based system, since it allows restriction enzyme independent cloning and leads to correct cDNA incorporation at almost 100% efficiency, circumventing the need to isolate and analyze large numbers of individual clones. Construct tagged libraries were prepared with the vector, pCMV5C, together with the FANTOM cDNA set obtained from RIKEN.

To generate modified clones, individual cDNAs (see below for selection criteria) were subjected to PCR amplification using low error rate Taq polymerase. cDNA-specific oligonucleotide primers were synthesized in house using a Gene Machines 96-well oligonucleotide synthesizer. PCR products were cloned into a pCMV5C ‘destination’ vector via an intermediary ‘entry’ vector using automated procedures. Simultaneous generation of an entry vector provides a resource for easy transfer into different destination vectors that encode alternative tags or direct bacterial or baculoviral protein expression. In the first round of screening, most of the cDNAs were tagged at the carboxy-terminus to allow transmembrane proteins and myristylated proteins to be targeted. However, for proteins that are modified at their carboxy-terminus, such as isoprenylated low molecular weight G-proteins, a variant of pCMV5C was used that introduces the epitope tag at the amino-terminus. Bacterial transformants were picked using a Colony Picker (Gene Machines, Inc.). For direct utilization, 10-20 colonies from each destination vector transformation were picked and pooled into a clone library. Use of a pool strongly reduces the risk that non-functional clones that might arise from PCR errors were used in the screen. Plasmid DNA from the pool was purified using commercially available automated vacuum manifold technology. Entry clones were fully sequenced and archived as part of a tagged sequenced signal transduction library set, which, is designated as a tagged signal transduction set (TST set).

Clone Selection.

To generate the flag-tagged TST, cDNAs of genes that encode proteins that possess domains known to be involved in signal transduction were selected. Analysis of the FANTOM set reveals approximately 40 modules contained in signal transduction and related proteins, as well as 200 cDNAs encoding cell cycle-related proteins. There is a particular interest in how signalling pathways are integrated at the level of the nucleus to control the transcriptional program of the cell. Therefore, the set of transcription factors and related molecules of which 200 are identified will be analyzed. To complete the set 112 proteins that possess identifiable PPI domains but otherwise are of unknown function will be included. Therefore, the initial version of the flag-tagged TST set encompasses approximately 1,000 proteins.

Defining Signal Transduction Interactomes

In vivo cells are exposed to multiple signals that are integrated to control cellular behaviour. How the signal transduction proteome, or more precisely, the interactome, fluxes in this complex environment is thus key to understanding how cellular behaviour is controlled in a physiological setting. The goal is to define a signal transduction interactome for four pathways that are critical regulators of cellular activity and proliferation. Preliminary experiments used Smad4 and TβRI as models for the development of the technology into a HTP format Thus, the initial focus will be on the TGFβ pathway and this will be expanded to screen and to explore the WNT, RTK and mitotic kinase pathways.

a) The TGFβ Interactome. To define interactomes studies will be conducted of the TGFβ pathway that signals through the core Smad pathway, which directly couples occupation of the TGFβ receptor with the transcriptional responses. Despite considerable knowledge about how Smads regulate gene responses to TGFβ, little is known of how other signalling pathways might be connected to the TGFβ receptor complex and how Smad binding to non-transcription factors, such as E3 ubiquitin ligases, might mediate TGFβ biology. Therefore, defining the TGFβ signalling interactome will yield important insights into how this pathway is involved in regulating diverse physiological and pathological processes.

The TGFβ signalling proteome is composed of approximately forty ligands, five type II and seven type I ser/thr kinase receptors, 8 Smad proteins, three SARA family proteins, 3 Smad-interacting proteins (STAM, Smurf1 and Smurf2) and 5 receptor-interacting proteins (TRAP, STRAP, TRIP, XIAP, and TAB1). In addition there are a host of DNA binding partners for Smads that mediate specific gene responses. Initially Smad proteins will be investigated and each Smad will be analyzed against the TST set in the presence and absence of TGFβ and BMP signalling. For the type II and type I receptors the interaction of wild type receptors or kinase-deficient variants, which can serve to stabilize the substrate-kinase interactions by ‘trapping’ the substrate will be examined, as was demonstrated previously for Smad2-TGFβ receptor interactions. In addition the BMP type II receptors, BMPRII and ActRIIB will be co-expressed together with the BMP type I receptors ALK2, ALK3 and ALK6 to examine the interactome of distinct receptor complexes. This is important because in vitro these receptor complexes recognize and activate Smad1, 5 and 8 with approximately equivalent kinetics, yet in vivo different BMP receptor complexes have very different biological functions. The unique functions of these otherwise closely related receptors may thus reflect activation of distinct downstream signalling pathways that are as yet undefined. Screening the BMPRII receptor is of particular interest as it has a unique carboxy-terminal extension that is mutated in human hereditary pulminary hypertension. Thus, the tail of BMPRII may play a critical role in coupling this receptor to unique pathways.

To conduct the screen the HTP protocol was used on the Thermo CRS integrated robotic platform. Briefly, cells were plated into 96-well tissue culture plates. 24 h after plating the cells in each well were transfected with a mix of pCMV5C that directs expression of a single luciferase-tagged Smad or receptor protein together with an individual flag-tagged cDNA from the TST set. Forty-eight hours after transfection, the cells were lysed. A small aliquot (10%) was removed to analyze total expression of the flag and luciferase-tagged proteins by a direct luciferase assay (Dual Glo™, Promega). A highly sensitive luciferase-based BLISA will also be used to analyze expression of the proteins. The remainder of the lysate was then subjected to immunoprecipitation using anti-flag M2 antibody and the immunoprecipitates collected and washed using protein G coupled to paramagnetic beads. The amount of luciferase that coprecipitates with the flag-tagged protein was then measured and quantitated relative to the total expression of the flag- and luciferase-tagged proteins. This approach allows a quantitative assessment of specific PPIs in the presence and absence of signalling.

b) The WNT Pathway. Wnt growth factors have been recognized to utilize two signalling pathways. The canonical pathway involves APC, β-catenin and Lef1/TCF, whereas the non-canonical pathway alters Ca⁺² mobilization. To explore the interactome of the WNT pathway the TST will be screened with luciferase-tagged components of the classical pathway (that include APC, β-catenin, axin, dishevelled, GSK-3β and TCFs1-4), as well as the various frizzled receptors, which are thought to preferentially activate either the classical or non-classical pathways. 293T cells display a robust response to Wnt signal transduction and are thus an excellent model for studying this pathway. Wnt ligands are not readily expressed in mammalian cells and tend to adhere to the matrix and it has been a challenge to generate soluble ligand. A cell line that secretes soluble and active Wnt3A, which stimulates the canonical pathway has been developed. Therefore, the TST set will be screened in the presence and absence of Wnt3A. Screens with the Wnt4 ligand and the appropriate frizzled receptors will also be conducted. These studies will better define the Wnt signal transduction pathway and identify components that regulate Ca⁺² mobilization.

c) SAK/Polo Pathway. The Sak/Plks play an important role in mitotic checkpoints that delay cell cycle progression in response to stress and DNA damaging agents (Sanchez, Y., Bachant, J., Wang, H., Hu, F., Liu, D., Tetzlaff, M., and Elledge, S. J. (1999). Science 286, 1166-1171; and Smits, V. A., Klompmaker, R., Arnaud, L., Rijksen, G., Nigg, E. A., and Medema, R. H. (2000). Nat. Cell Biol. 2, 672-676). The catalytic domains, and the motifs that regulate subcellular localization as well as protein stability are dependent on PPIs, which are numerically and temporally complex. For example, the polo box motif has been defined in Sak kinase, as necessary and sufficient to localize the enzyme to the nucleolus during G2, to the centrioles in G2/M, and at the actin-cleavage ring during telophase. The various locations of Plks in the cell suggest there are different binding partners for localization and different substrates. The yeast homolog (Cdc5) is known to interact with more than 10 proteins (cyclin B1, Scc1, APC-Cdc20, α-β-γ-tubulin, Lte1, Bub2, septins, MKLP-1, Mid1p, Hsp90). The PPI network in which mammalian Plks function are not yet well understood. The PPI screen will be performed using Plks, and in cells under different experimental conditions to identify cell-cycle and stage specific interactomes. For this, immortalized NIH-3T3 cells and HeLa tumor cells will be used. The cells will be either growing asynchronous, blocked in M phase with nocodazole, or blocked in G2 phase with thymidine/aphidicolin. Checkpoints will also be imposed; the spindle checkpoint (M with nocodazole for 8 h), the microfilament checkpoint (10 μM latrunculin B for 8 h) and the DNA damage checkpoint (nocodazole for 8 h, then for 1 h with 0.5 μM of adriamycin). Understanding how the interactome of mitotic kinases changes during cell cycle progression will assist in identifying new genes that may be causal in cancer, and will suggest new targets for cancer therapy.

d) RTK Pathways. Elucidation of how specific cellular responses are elaborated is of primary importance in biology. In principle, specificity could come from signals that activate pathways dedicated to specific responses. However, most RTKs can activate the same core signalling mediators, so how specific cellular responses are elicited is unclear. Preferential activation of certain components of these signalling networks by different receptors might direct the cellular response. Amplitude and timing of pathway activation may also play a key role in this process. For instance, transient activation of RTK pathways in PC12 cells fails to induce differentiation whereas extended activation promotes neurite outgrowth. Along these lines, cell commitment to RTK signals requires 6 to 8 hours of treatment despite the fact that all of the known early signalling events have been completed within an hour or two (Hunter, T. (2000). Cell, 100, 113-127). The systematic and quantitative analysis of all PPIs that mediate RTK responses is thus essential to address this important biological question.

To examine this, a functional genomics approach will be applied to define how the RTK signal transduction interactome fluxes in both time and in response to activation of distinct receptors. Approximately 100 proteins have been identified within the RIKEN set that comprise the key pathways mediating RTK responses. Thus, screens will be conducted in which the interaction between flag- and luciferase-tagged version of each of these proteins is examined. This yields a matrix of 100 by 100, or 10,000 individual protein-protein interactions.

The automated HTP screen is ideally suited for these types of studies, which would otherwise be extremely challenging in a typical laboratory. The EGF pathway will be examined using a model cell system, 293T cells, which has abundant EGF receptors, and displays a robust response to EGF. Cells will be treated with EGF for varying lengths of time and PPIs at each time point defined. For initial studies, time points of 0, 5 min, 1 hr, and 8 hr will be used so that early, intermediate and later signalling events will be revealed. To compare how the interactome varies in response to different activators these screens will be repeated using different ligands including FGF, PDGF, NGF and soluble Ephrins. These screens will define how the common signalling networks downstream of RTKs translate different signals into distinct cellular responses.

The results can be deposited in a database (e.g. Biomolecular Interaction Network Database (BIND)—http://www.bind.ca: Bader G D et al, Nucleic Acids Res 2001, 29: 242-245) and subjected to bioinformatic analysis. Based on the pilot screens with TβRI numerous novel PPIs will be identified. As some of these may not reflect physiological associations, it will be important to validate novel interactions using endogenous proteins.

The Interactome of Novel Proteins.

One of the key challenges facing the recent decoding of the human genome is understanding the function of proteins encoded by novel genes. When the primary sequence of these protein products is highly conserved with other proteins of known function, putative functions can be inferred. However, in many cases the gene products have only weak similarity to known genes and this is often restricted to specific domains. Understanding the interactome of unknown proteins provides critical clues that can greatly accelerate the process of understanding their function. For this aspect, 20 proteins that have recognizable domains but otherwise have unknown function will be examined. In selecting this set LKB1(STK11), TUBEROUS SCLEROSIS 1 and 2 (TSC1 and 2) and POLYCYSTIC KIDNEY DISEASE 1 and 2 (PKD1 and 2) have been selected as model case studies. LKB1 is a tumour suppressor gene that is mutated in Peutz-Jeghers syndrome, a disease characterized by intestinal hamartomas and increased risk of cancer (21,22). The product of the LKB1 gene is an intracellular ser/thr kinase of unknown function. Mutations in TSC1 and TSC2 cause tuberous sclerosis, which is a hyperproliferative disease of soft tissue that leads to the formation of hamartomas (23). The TSC proteins possess coiled-coiled protein-protein interaction domains and have been shown to inhibit insulin signalling, but the molecular mechanisms are undefined. Finally PKD1 and PKD2 are mutated in almost all human polycystic kidney disease. They are predicted to encode components of a membrane protein complex that can activate a number of signalling cascades, such as PKC and β-catenin, however, the downstream components that connect these novel membrane proteins to intracellular signalling networks is unknown (24). The ability to screen large numbers of protein-protein interactions will help place these and other putative signalling proteins into specific pathways.

To map the interactome of these gene products, it is technically feasible to screen each of the selected proteins against the entire TST set in the presence and absence of a broad range of extracellular factors such as TGFβ, EGF, WNT, hedgehog, TNFα, etc. However, it is possible that in the absence of signalling these proteins may display interactions with key components of known pathways that would provide an important clue as to their function. Thus, a more directed approach will first be used. For this, each of the proteins will be screened against the entire TST set in the absence of specific extracellular stimulae. Based on this initial screen, interactions that are detected with key components of known signalling pathways may suggest which extracellular stimulae to focus on for subsequent induction screens. These studies will provide major insight into the function of novel gene products and may provide treatment targets where mutations in the gene are causative of human disease.

Integration and Analysis of the Interactome Datasets.

The HTP screens will generate considerable amounts of raw data. An information management system can be developed in which the results of specific PPI tests are summarized in a PPI report. The PPI report describes the magnitude of the interaction, the stimulus used and includes links to the raw data (cell type, protein expression levels and robotics logs) used to generate the report. This will be achieved through web-based deposition using for example the BIND specification. The tools developed for BIND will enable visualization of the results of the screen and link these results to web-based resources. Visual representation of the screen will take the form of objects representing proteins connected by lines that are representative of specific interactions. Therefore, each PPI will be screened against the databases of interacting proteins and PUB-MED® for confirmation and potentially novel interactions validated by conventional methods.

Interactome mapping efforts thus far have focussed on the yeast, which was the first genome to be fully sequenced (Tucker, C. L., Gera, J. F., and Uetz, P. (2001). Trends Cell Biol. 11, 102-106). These approaches have relied almost exclusively on yeast two-hybrid methods and have generated enormous amounts of information. In contrast, bioinformatics approaches, exemplified by the DIP, BIND and TRANSpath database systems have relied on culling reports from the published literature of protein-protein interactions. Together these approaches have generated complex interaction networks that provide a descriptive record of protein-protein interactions. From these analyses however, it is difficult to derive an understanding of how cellular behaviour is controlled by extracellular signals in a physiological environment. This is primarily because these approaches are devoted to defining interactions in a binary manner, that is, interactions are recorded as simply present or not present. However, extracellular stimulae act to dynamically regulate the magnitude of PPIs in both space and time, in large part through alterations in the postranslational modification of key signalling proteins. Therefore, to understand how the signal transduction proteome controls net cellular behaviour it is essential to understand how the interactome fluxes in mammalian cells in response to stimulation and environment For this it is essential to obtain quantitative and not simply qualitative data. The approach described herein permits a quantitative assessment of PPI flux in mammalian cells in response to external stimulae. Quantitative information for interactions can be colour-coded as shown in FIG. 3 and tools can be developed to encode dynamic changes that occur upon growth factor treatment. More complex analysis such as clustering may be implemented, which will compare all data to extract those PPIs that display similar dynamics and thus may function as part of a coordinated response. Finally, it will be noted where PPIs lead to cell behaviour or gene responses. For instance, interactions with DNA binding partners will be linked to specific gene responses, whereas association with components of cytoskeleton remodelling will be linked to cell motility and polarity. This latter aspect of data analysis will allow modeling of how the signal transduction interactome is integrated in complex environments to regulate net cellular activity. In particular, quantitative analysis of PPIs is a critical resource for mathematical modelling of cellular behaviour.

### EXAMPLE 2

HTP Method for the Detection of Protein-Protein Interactions in Mammalian Cells.

HEK-293T (human endothelial kidney) cells were maintained in DMEM (Dulbeco's Modified Eagle's Medium) supplemented with 10% fetal bovine serum at 37° C. The cells were plated in COSTAR (Corning, N.Y.) Poly-D-Lysine-coated 96-well flat bottom tissue culture plates at a density of 20,000-24,000 cells per well at least 18 h prior to transfection in the Robotic platform.

Cells were transfected via PolyFect (QIAGEN, Hilden, Germany) with a total of 200 ng of DNA per well, 100 ng corresponding to the luciferase-tagged proteins (see below) and the other 100 ng corresponding to a flag-tagged cDNA from the TST set (see below) with or without a receptor from the TGF-β family. The cells were maintained at 37° C. for 48 h after transfection and then lysed for 15 min with a 0.5% Triton-X containing buffer in the presence of protease and phosphatase inhibitors. A small aliquot was removed (10%) and transferred to a COSTAR round bottom white plate to determine the total expression levels of the luciferase-tagged proteins using the Dual-Glo Luciferase assay system from Promega Corporation (Madison, Wis., USA). The remainder of the lysate was then mixed with Protein G-paramagnetic beads (Dynal, Oslo, Norway), coupled with the anti-flag M2 antibody (Sigma-Aldrich, St. Louis, USA) previously dispensed in a Non-Binding Surface round bottom white plate (COSTAR). The cell lysate and magnetic beads mixture was incubated at 4° C. for 1 h followed by eight washes with the aid of a custom-made magnet. The amount of luciferase-tagged protein that co-precipitates with the flag-tagged protein was then measured with the Dual Luciferase assay system from Promega. The amount of luciferase activity in the precipitates relative to the total expression levels allowed a quantitative assessment of specific PPIs in the presence and absence of signaling.

Tagged Signal Transduction cDNA Set (TST Set).

1,000 cDNAs containing domains known to be involved in signal transduction were selected from the FANTOM set provided by RIKEN. 680 were subjected to PCR amplification and subcloned into the customized topoisomerase-based pCMV5C vector. 560 clones tagged at the N-terminus were obtained. The expression of these clones in mammalian cells was confirmed by immunofluorescence.

Defining Signal Transduction Interactomes.

A collection of Renilla luciferase (Rluc) fusion proteins was constructed for the TGF-β interactome. The members of the TGF-β pathway that have been tagged with Rluc are as follows:
a) SMADS: SMAD2 (R-Smad), SMAD4 (Co-Smad) and SMAD7 (I-Smad);
b) TGF-β Receptors: TGF-βReceptor Type I wild type [TβRI wt], TGF-βReceptor Type I kinase deficient [TβRI (K/R)] and TGF-βReceptor Type I constitutively active [TβRI (T/D)];
c) BMP-7 Receptors: ALK-2 wild type [ALK-2 wt] and ALK-2 constitutively active [ALK-2 (Q/D)];
d) BMP-2 Receptors: ALK-6 wild type [ALK-6 wt], ALK-6 constitutively active [ALK-6 (Q/D)], and ALK-6 kinase deficient [ALK-6 (K/R)];
e) SMURFs: SMURF2 constitutively inactive [SMURF2 (C/A)].
   Automated Platform to Detect PPIs in Mammalian Cells.

Each one of the members of the TGF-β pathway described above was tested against the 560 TST set To accomplish this, the assay described above was standardized on an integrated robotics platform developed with ThermoCRS, Burlington, Ontario, Canada. This platform consists of a Catalyst 5 robotic arm on a 3 m rail which is controlled by POLARA scheduling software. On each side of the rail the arm has access to the following instruments:

Cell Culture:

HOTPACK CO₂ incubator with capacity for 120 plates

Liquid Handlers:

Beckman Multimek with a 96-channel head for disposable tips

Thermo Labsystems Multidrop with 8-channels

Packard Multiprobe with 8-channels

Biotec ELx405 Washer with a 96-channel head and integrated magnet

Readers:

Molecular Devices CLIPR

Molecular Devices Spectramax Plus

Molecular Devices Spectramax Gemini

Microtitre Plate Handling and Storage:

Carousel with capacity for 40 disposable tip boxes or 120 microtitre plates

Lidding station

Four Platefeeders with capacity for 60 microtitre plates or 20 tip boxes

Shaker incubator at 4° C. with 20 microtitre plate-capacity

Velocity 11 Bar-code print and apply

Microscan Bar-code reader

Re-Grip station

CRS Magbead hotels

Off-line access to a EG&G Berthold Microlumat Plus Luminometer for 96-well plate format.

Magna washer and the CRS Magbead

The results of the interaction assays for TGFβ are shown in Tables 1 and 2. Table 1 lists previously known interactions present in the TST set Table 2 lists novel interactions found in the HTP screen.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1
List of previously known interactions present in the
TST set found during the HTP screen.

| BAITS | Confirmed Known Interaction Partners |
|---|---|
| SMADS | |
| SMAD4 | |
| SMAD4 + TGF-β | SMAD2, SMAD3 |
| SMAD2 | SMURF2 (C/A), Ski |
| SMAD2 + TGF-β | SMURF2 (C/A), Ski |
| SMAD7 | SMURF2 (C/A) |
| SMAD7 + TGF-β | SMURF2 (C/A) |
| Receptors | |
| TGF-β | |
| TβRI (T/D) | TβRI, FKBP12 |
| TβRI (K/R) | TβRI, FKBP12 |
| TβRI (WT) | |
| BMP-7 | |
| ALK2 (Q/D) | TβRI |
| ALK2 (WT) | TβRI |
| BMP-2 | |
| ALK6 (Q/D) | BMPRII, TβRI |
| ALK6 (K/R) | TβRI |
| ALK6 (WT) | TβRI |
| SMURFS | |
| SMURF2 (C/A) | SMAD1, SMAD3, SMAD7, SMAD6 |

TABLE 2
Novel Interactions found during the HTP screen
for protein-protein interactions.

| BAIT | cDNA ID |
|---|---|
| SMADS | |
| SMAD4 | 1-81, 1-77, 1-70, 5-51 |
| SMAD4 + TGF-β | 1-81, 1-77, 1-70, 5-51 |
| SMAD2 | 5-60, 5-66, 2-75 |
| SMAD2 + TGF-β | 3-49, 2-80 |
| SMAD7 | |
| SMAD7 + TGF-β | 2-80 |
| Receptors | |
| TGF-β | |
| TβRI (T/D) | 1-83, 1-88, 5-34, 5-67, 5-75, 5-78, 7-58, 3-43, 3-49 |
| TβRI (K/R) | 1-83, 1-88, 5-34, 5-67, 5-78, 7-58, 4-62 |
| TβRI (WT) | 1-83, 1-88, 5-34, 5-75, 5-78, 3-49 |
| BMP-7 | |
| ALK2 (Q/D) | 5-20 |
| ALK2 (WT) | 5-20, 3-41, 3-49 |
| BMP-2 | |
| ALK6 (Q/D) | 5-75 |
| ALK6 (K/R) | 1-83, 1-88, 5-46, 7-35, 3-43, 4-25 |
| ALK6 (WT) | 1-83, 1-88, 5-75, 3-43 |
| SMURFS | |
| SMURF2 (C/A) | 1-25, 1-34, 1-81, 2-80, 7-16, 4-25 |

### REFERENCES

1. Attisano, L., and Wrana, J. L. (2000). Curr. Op. Cell Biol. 12, 235-243
2. Wrana, J. L., and Attisano, L. (2000). Cyto. Growth Factor Rev. 11, 5-13
3. Massagué, J., Blain, S. W., and Lo, R. S. (2000). Cell 103, 295-309
4. Cadigan, K. M., and Nusse, R. (1997). Genes Dev. 11, 3286-3305
5. Kuhl, M., Sheldahl, L. C., Park, M., Miller, J. R., and Moon, R. T. (2000). Trends Genet. 16, 279-283
6. Hunter, T. (2000). Cell, 100, 113-127
7. Pawson, T., and Saxton, T. M. (1999). Cell 97(97), 675-678
8. Pawson, T., and Nash, P. (2000). Genes Dev. 14, 1027-1047
9. Sanchez, Y., Bachant, J., Wang, H., Hu, F., Liu, D., Tetzlaff, M., and Elledge, S. J. (1999). Science 286, 1166-1171
10. Smits, V. A., Klompmaker, R., Arnaud, L., Rijksen, G., Nigg, E. A., and Medema, R. H. (2000). Nat. Cell Biol. 2, 672-676
11. Pennisi, E. (1998). Science 279, 477-478
12. Hudson, J. W., Kozarova, A., Cheung, P., Macmillan, J. C., Swallow, C. J., Cross, J. C., and Dennis, J. W. (2001). Curr. Biol. 11, 441-446
13. Tucker, C. L., Gera, J. F., and Uetz, P. (2001). Trends Cell Biol. 11, 102-106
14. Pollok, B. A., and Heim, R. (1999). Trends Cell Biol. 9, 57-60
15. Remy, I., and Michnick, S. W. (2001). Proc. Natl. Acad. Sci. (USA) 98, 7678-7683
16. Michnick, S. W. (2001). Curr. Op. Struct. Biol. 11, 472-477
17. Rossi, F., Charlton, C. A., and Blau, H. M. (1997). Proc. Natl. Acad. Sci. USA 94, 8405-8410
18. Figeys, D., McBroom, L. D., and Moran, M. F. (2001). Methods 24, 230-239
19. Kim, S. K. (2000). Nat. Cell Biol. 2, E143-145
20. Kawai, J., et al., (2001). Nature 409, 685-690
21. Hemminki, A., Markie, D., Tomlinson, I., et al., (I998). Nature 391, 184-187
22. Jenne, D. E., Reimann, H., Nezu, J., Friedel, W., Loff, S., Jeschke, R., Muller, O., Back, W., and Zimmer, M. (1998). Nat Genet. 18, 38-43
23. Cheadle, J. P., Reeve, M. P., Sampson, J. R., and Kwiatkowski, D. J. (2000). Hum Genet. 107, 97-114
24. Peterson, R. T., and Schreiber, S. L. (1999). Curr Biol. 9, R521-524

1. (canceled)

2. (canceled)

3. A method for quantitating protein-protein interactions which method comprises the steps of:
(a) expressing one or more prey protein in cells, wherein a prey protein is labelled with an epitope tag permitting separation of the prey protein from other proteins in the cells;
(b) expressing one or more bait protein in the cells wherein a bait protein is labelled with a detectable substance permitting identification of the bait protein and protein-protein interactions comprising a prey protein and the bait protein;
(c) obtaining a lysate of the cells and assaying an aliquot of the lysate to measure total expression of the epitope tag and detectable substance;
(d) assaying a second aliquot of the lysate to measure the amount of a detectable substance that coprecipitates with an epitope tagged prey protein; and
(e) comparing the amounts measured in steps (c) and (d) to quantitate the protein-protein interaction.

4. A method as claimed in claim 3 wherein the cells are subjected to an extracellular or intracellular signal after step (b).

5. A method for determining an interactome for one or more bait protein comprising:
(a) preparing recombinant cells each expressing one or more bait protein and one or more prey protein selected from a variegated population of prey proteins;
(b) inducing formation of protein-protein interactions between a prey protein and bait protein in the cells;
(c) identifying protein-protein interactions comprising a prey protein and bait protein to thereby determine the interactome for the bait protein.

6. (canceled)

7. (canceled)

8. (canceled)

9. (canceled)

10. (canceled)

11. (canceled)

12. A method for assaying for changes in protein-protein interactions in response to intracellular or extracellular factors or a test agent comprising:
(a) introducing one or more prey protein in cells, wherein a prey protein is labelled with an epitope tag permitting separation of the prey protein from other proteins in the cells;
(b) introducing one or more bait protein in the cells, wherein a bait protein is labelled with a detectable substance permitting identification of the bait protein and protein-protein interactions comprising a prey protein and the bait protein;
(c) inducing formation of protein-protein interactions between a prey protein and bait protein;
(d) introducing an intracellular or extracellular factor or test agent;
(e) assaying protein-protein interactions comprising a prey protein and bait protein; and
(f) comparing the assayed protein-protein interactions with protein-protein interactions assayed in the absence of the intracellular or extracellular factor.

13. (canceled)

14. A method of claim 12 wherein an increase in the protein-protein interactions with a test agent indicates that the agent is an agonist of the interaction and a decrease in the amount of protein-protein interactions indicates that the agent is an antagonist.

15. A method of claim 5 wherein the cells are mammalian cells.

16. A method as claimed in claim 5 wherein one bait protein is introduced or expressed in the cells.

17. A method as claimed in claim 5 wherein two or more bait proteins are introduced or expressed in the cells.

18. A method as claimed in claim 17 wherein each bait protein is labeled with a different detectable substance.

19. A method as claimed in claim 5 wherein the detectable substance is an enzyme, radioisotope, fluorescent label, luminescent label, or an enzymatic label.

20. A method of claim 19 wherein the detectable substance is an enzymatic label.

21. A method of claim 20 wherein the detectable substance is luciferase, in particular Renilla luciferase.

22. A method as claimed in claim 17 wherein two or more prey proteins are introduced into the cells.

23. A method of claim 17 wherein the epitope tag is FLAG, hemagglutinin, His6, or an Ig sequence.

24. A method of claim 5 wherein the prey protein comprises a protein sequence obtained from genomic DNA sequences or random sequences.

25. A method of claim 5 wherein the prey protein comprises a library of protein sequences.

26. A method of claim 5 wherein the bait protein is a functional domain of a protein involved in signal transduction.

27. A method of claim 5 wherein the bait protein is a protein of the TGFβ proteome, Wnt/Wingless pathway, Sak/Polo pathway, or a receptor tyrosine kinase pathway.

28. A method of claim 5 wherein the bait protein is a Smad protein, SARA family protein, Smad-interacting protein, TGFβ receptor, TGFβ receptor interacting protein, SMURF, BMP receptor, APC, β-catenin, axin, dishevelled, GSK-3β, TCFs1-4, Sak, Plks, EGF, FGF, PDGF, or NGF.

29. (canceled)

30. (canceled)

31. (canceled)
